(19) United States
(12) Patent Application Publication
(10) Pub. No.: US 2005/0033251 A1
(43) Pub. Date: Feb. 10, 2005

(54) Controlled release of biologically active substances from select substrates

(75) Inventors: William Toreki, Gainesville, FL (US); Gregory Staab, Gainesville, FL (US); Gerald Olderman, Bedford, MA (US)

Correspondence Address: ELMAN TECHNOLOGY LAW, P.C., P. O. BOX 209, SWARTHMORE, PA, 19081-0209, US

(73) Assignee: Quick-Med Technologies, Inc., Gainesville, FL (US)

(21) Appl. No.: 10/786,959

(22) Filed: Feb. 25, 2004

Related U.S. Application Data
(63) Continuation-in-part of application No. PCT/US02/30998, filed on Sep. 30, 2002.
Application No. PCT/US02/30998 is a continuation-in-part of application No. 09/965,740, filed on Sep. 28, 2001.
Application No. 09/965,740 is a continuation-in-part of application No. PCT/US99/29091, filed on Dec. 8, 1999.
(60) Provisional application No. 60/111,472, filed on Dec. 9, 1998.

(51) Int. Cl. A61F 13/15; A61F 13/20

(52) U.S. Cl. 604/367

(57) ABSTRACT

This invention relates to methods and compositions for materials having a non-leaching coating that has antimicrobial properties. The coating is applied to substrates such as gauze-type wound dressings, powders and other substrates. Covalent, non-leaching, non-hydrolyzable bonds are formed between the substrate and the polymer molecules that form the coating. A high concentration of anti-microbial groups on multi-length polymer chains and relatively long average chain lengths, contribute to an absorbent or superabsorbent surface with a high level antimicrobial efficacy. Utilization of non-leaching coatings having a plurality of anionic or cationic sites is used according to this invention to bind a plurality of oppositely charged biologically or chemically active compounds, and to release the bound oppositely charged biologically or chemically active compounds from said substrate over a period of time to achieve desired objectives as diverse as improved wound healing to reduction in body odor.

### CROSS-REFERENCE TO RELATED APPLICATIONS

This application is a continuation-in-part of our co-pending Application, Serial Number PCT/US02/30998, filed Sep. 30, 2002, which itself was a continuation-in-part of co-pending U.S. patent application Ser. No. 09/965,740, filed Sep. 28, 2001, which itself was a continuation-in-part of our co-pending PCT application Ser. No. PCT/US/99/29091, filed Dec. 8, 1999, pending, which is a continuation of U.S. Provisional application Ser. No. 60/111,472, filed Dec. 8, 1998, now abandoned, to which the benefit of priority is claimed under 35 USC §§ 119 and 120. The PCT/US/99/29091 application is hereby incorporated by reference, as is the disclosure of the Ser. No. 09/965,740 application.

### BACKGROUND OF THE INVENTION

The importance of sterile techniques and especially sterile bandage material to modem medicine can hardly be overestimated. Almost every student of biology has heard the tales of how medical practitioners of not too long ago thought that pus and other signs of what is now known to be infection were essential to wound healing. These practitioners would reopen a wound that was not showing the expected pus and inflammation. This was changed by Lister's discoveries regarding disinfection and the subsequent adoption of sterile bandage material for wound dressings. A continuing problem has been the propensity for microorganisms to grow in once sterile bandage material.

A major function of surgical bandages and packing materials is the absorption of various excreted fluids. These fluids are frequently rich in nutrients and are capable of supporting abundant bacterial growth. Since the surgical opening or skin surface is rarely absolutely free of bacteria, the bandage material soon supports a burgeoning bacterial population. These bacteria can easily cause serious infection and may also release a variety of harmful toxins. The obvious solution to such a problem is to change the bandage material often so that bacterial buildup does not occur. An additional approach is to treat the bandage material with some type of disinfectant to limit bacterial growth. Unfortunately, it has proven difficult to produce an effective disinfectant that does not readily wash out of the material. Such wash out, or leaching, reduces effectiveness and may cause irritation or damage to body tissues.

This problem is not limited to bandages, dressings or packings for wounds or surgical incisions. There are a number of instances where absorptive packings are placed in natural body orifices with significant possibility for dangerous bacterial growth. Various nasal packings can become bacterially laden following insertion into nasal passageways. Numerous deaths have resulted from “toxic shock syndrome” resulting from multiplication of Staphylococcus aureus bacteria in feminine care products, particularly tampons. There have been a large number of related problems. For example, U.S. Pat. No. 5,641,503, to Brown-Skrobot, seeks to produce a germicidal tampon and contains a useful list of references to the toxic shock problem. A particular difficulty has been that many potent germicidal agents, e.g. iodine, are partially or totally ineffective in the presence of protein rich solutions such as blood or menses.

In addition, due to the globalization of commerce, the emergence of new diseases, the risks of biological warfare, the contamination of food products with highly pathogenic strains of bacteria, and other forces on society, the uses for a technology that imparts a non-leaching antimicrobial coating to a variety of surfaces is duly recognized. Specific areas of use in addition to bandage material are described below in the Summary of the Invention section.

In connection with the care and treatment of wounds, the term “wound” is meant to include burns, pressure sores, punctures, ulcers and the like. For a long time, one critical aspect of wound care has been the consideration of the requirements of the epithelium, i. e., that area of new cell growth directly peripheral to the wound which is formed during the healing process, so that healing is facilitated.

Since it has been recognized that healing of the wound occurs in one sense as the epithelium migrates by growth from the periphery inward, care has been taken not to damage unnecessarily or to irritate this new area of growth or existing, compromised periwound tissue. With many dressings, problems can occur during dressing changes. This is particularly true where the dressing adheres to the epithelium or where granulation tissue and new cell growth become intertwined within the matrix of a dressing. In these instances, there is a risk that removal of the dressing will damage the sensitive tissue and new growth on the periphery of the wound thereby causing a regression in the progress of wound healing.

Another consideration in wound care is the frequency of dressing changes. The time frame for the changing of dressings depends on many concerns and therefore opinions as to how often dressings should be changed vary drastically.

Still, another important consideration in wound care is the needs of the surrounding unwounded skin. The unwounded skin beyond the epithelium is usually in contact with some portion of the wound dressing system which maintains the dressing positioned on the wound. For example, the surrounding skin may be covered for extended periods with a wrap and/or adhesive to hold the dressing in place. Many such dressings can irritate this surrounding skin and compound problems to the patient. This is especially true in the area of leg ulcers wherein the surrounding skin can easily become sensitized by strong medicaments and is often plagued with flaking, scaling and eczema.

One type of treatment presently used, in particular for leg ulcers, comprises the application of gauze to the ulcer and the utilization of a compression wrap to secure the gauze to the ulcer. Since the gauze quickly becomes saturated, frequent changes are necessary and damage to the epithelium and surrounding skin may occur. Moreover, if the gauze is left on for too long a period, the exudate can begin to overly hydrate and macerate the patient's surrounding skin.

A second type of treatment, also used in particular for leg ulcers, is the Unna's Boot (commercially available from Biersdorf, Inc.) which comprises a zinc paste-containing bandage wrapped around a patient's leg from above the toes to below the knee. Other Unna's Boot/zinc impregnated treatments are available from Miles and Graham Field. These dressings are typically left in place for a week at a time and absorbent pads must be applied to the outside of the dressings in the area of the ulcer to absorb excess exudate. Seepage of exudate throughout the wrap is common, and damage to the skin and epithelium is inevitable.

Another type of wound dressing is disclosed in U.S. Pat. No. 5,106,362 to Gilman. This dressing is provided with a base sheet for contacting the skin of a patient. The base sheet has an opening for placement over the wound. The dressing has a vent for providing controlled leakage of fluid along a path from the wound through the opening of the base sheet. The vent is designed to provide control over wound leakage along a “tortuous path” from the wound through the opening of the base sheet.

A modification of the dressing of U.S. Pat. No. 5,106,362 is disclosed in U.S. Pat. No. 5,056,510, also to Gilman. The '510 patent discloses a vented dressing where the fabric reservoir for wound exudate is contained within a chamber. The walls of the chamber are intended to provide a barrier to bacterial and other contaminants. The walls of the chamber are also intended to be air permeable so as to permit egress of air from the voids of the fabric reservoir. These Gilman dressings do not especially address the problems of the epithelium and the surrounding skin.

It is apparent that, considering the various types of wounds, the numerous dressings that are available, and the various stages of healing, there is still a tremendous need for a dressing that functions better than the current dressings, especially with respect to preventing damage to surrounding skin, tissue and new cell growth. In particular, a wound dressing system which protects the epithelium and surrounding non-wounded skin, which wicks away moisture from the wound area, and which does not purposely adhere to the wound or the surrounding area would be a useful addition to the wound care art. A dressing for patients with fragile skin surrounding a wound would be especially beneficial.

Certain wound dressing materials have been used to absorb exudate and promote healing. For example, Mason, et al., U.S. Pat. No. 4,393,048 teaches a hydrogel composition which, when applied as a powder, absorbs wound exudate. The hydrogel formation may not be complete and lumps of partially hydrated powders form which, when removed, may reopen the wound.

It is known that wounds heal more rapidly and completely if kept in a slightly moist or hydrated state. Polyethylene glycol containing hydrogel wound coverings are disclosed in U.S. Pat. No. 4,226,232, to Spence. These hydrogels cannot be sterilized by irradiation due to the formation of free radicals.

Rawlings et al., U.S. Pat. No. 4,657,006, illustrates wound dressings comprised of a hydrophilic polymer having moisture and vapor permeability properties. However, the exudate absorbed by the hydrophilic polymer tends to harden or solidify the polymer.

An ideal wound dressing should not only absorb exudate but also possess antimicrobial or antibacterial properties. As used in this disclosure, “antibacterial” is defined as having an adverse effect on bacteria, particularly disease-causing bacteria. Furthermore, “antimicrobial” is defined as having an adverse effect on a range of microorganisms, including bacteria and at least some fungi and viruses. An antimicrobial wound dressing is generally preferred over an antibacterial wound dressing.

One example of an antimicrobial wound dressing is Matson, U.S. Pat. No. 4,728,323, which discloses a wound dressing comprising a substrate coated with a coating of a silver salt that allegedly also keeps the wound moist. Since the active agent (silver ion) is not covalently bound to the dressing material, there is a potential for leaching into the body and/or depletion of the active agent.

In the past, wounds have been treated with antimicrobial active agents applied to the wound and covered with a covering that inhibits the healing process. For example, it was conventional practice early in the 20th Century to apply an antiseptic mercury agent such at thimerosal (Merthiolate) or merbromin (Mercurochrome) and the like to a wound and then cover or wrap the wound with a bandage such as gauze or an adhesive strip having a central absorbent gauze portion. A disadvantage of this approach is that the wound often weeps or exudes fluids such as blood, pustulation and the like. While the gauze may absorb some of these fluids, the gauze often adheres to the wound such that removal of the dressing reopens the wound. Advances in the art have been made in both bandages and antimicrobial agents. Certain bandages now contain a nonadhering polymeric coating over or, in place of, the gauze that inhibits the adhering of the absorbent material to the wound but also inhibits the absorption of the exudate that is necessary to properly heal the wound.

Korol, U.S. Pat. No. 4,563,184, discloses wound dressings comprising a polymer, such as poly(2-hydroxyethylmethacrylate), a solvent, such as polyethylene glycol, and a plasticizer such as DMSO. An antimicrobial agent, such as silver sulfadiazine, may be incorporated into the polymeric material.

Widra, U.S. Pat. No. 4,570,629, is drawn to absorbent hydrogel membrane wound dressings made up of hydrophilic biopolymeric copolyelectrolytes comprising a water-soluble linear anionic protein polyelectrolyte component derived from keratin and a water-soluble linear cationic biopolymer polyelectrolyte component derived from either collagen or a glycosaminoglycan. The membranes may also contain antibiotics.

Klemm et al., U.S. Pat. No. 4,191,743, teach the administration of antibiotics to wounds using a wound dressing comprising at least two layers of synthetic resin arranged one above the other having an intermediate layer composed of a synthetic resin granulate having an antibiotic incorporated therein.

Hansen et al., U.S. Pat. No. 5,498,478 is directed to the use of a polyethylene glycol or similar polymer as a binder material for fibers of any variety. The binder and fibers may be pretreated by slurrying the fibers in baths containing antimicrobial agents as part of the solution, thereby causing the fibers and the subsequently formed matrix of polymer and fibers to have an antimicrobial ability.

Mixon et al., U.S. Pat. No. 5,069,907 is directed to the formation and use of a polymeric sheet which may include an antimicrobial agent. This patent teaches the inclusion of antimicrobial agents into either a pressure-sensitive layer, such as an adhesive, or in a drape used to cover a wound or other sensitive area.

Dietz et al., U.S. Pat. Nos. 5,674,561 and 5,670,557 are directed to polymerized microemulsion pressure sensitive adhesive compositions that may optionally contain antimicrobial and/or other biologically active agents. The potential antimicrobial activity of quaternary amine and quaternary ammonium salts is taught. It is further taught that an antimicrobial agent can be added so as to be contained in a specific layer of a pressure sensitive adhesive device for use as a medical skin covering and/or as a wound dressing.

Young et al., U.S. Pat. No. 5,432,000, teach the use of a polymeric network for adhering particulate materials to a fiber or fibrous product. Specifically, this patent teaches the use of polymers, such as polyethylene glycol or polyethylene to cause the binding of particulate materials to a fiber, such as cloth. One such particulate member which could be adhered to cloth is an antimicrobial agent, such as epoxide phenol or another antimicrobial substance.

U.S. Pat. No. 5,811,471, to Shanbrom, teaches immobilization of germicidal dyes such as methylene blue onto polyvinyl alcohol gels. The dyes are not covalently bound; however, and thus have the potential to be desorbed. This potential shortcoming is discussed in '471: “Even though dyed PVA appears non-irritating, there might be some concern that the disinfectant dye molecules could migrate to human tissue in contact with the material”. Another potential drawback discussed therein is that the dyes are strongly colored, and hence may not be visually appealing to the consumer: “In the case of some products like tampons a “clean” white product might be psychologically more acceptable”.

In U.S. Pat. No. 4,643,181, Brown discusses mixing an antimicrobial biguanide compound with an adhesive. The adhesive polymer is incorporated into the system to bind the biguanides (which are desorbed from the non-woven material when it is wetted by urine. It is clear from the data that the biguanide antimicrobial leaches from the material, and thus it is a drug-releasing system.

A number of antimicrobial systems based on leaching of low concentrations of silver ion from surfaces have also been reported. For instance, U.S. Pat. Nos. 6,126,931 and 6,030,632 describe a biguanide polymer (PHMB) bonded a substrate. Silver salts are then bonded to the immobilized PHMB. The surface-bound PHMB alone does not inhibit bacterial growth, but it does bind the bacteria, thus allowing the low-solubility silver salts to function.

A similar invention is reported in U.S. Pat. No. 5,662,913, to Capeli, wherein wound dressings which contain silver salts are discussed. The silver is stabilized by polyether polymers.

Another similar method is described in U.S. Pat. No. 5,856,248, to Weinberg, except that copper salts are used instead of silver.

The leaching of silver from elemental silver coatings on medical textiles is described by Tweden et al. (“Silver Modification of Polyethylene Terephthalate Textiles for Antimicrobial Protection”; ASAIO Journal, 43, pM475-M481 (1997). In that study the leaching of silver was equivalent to a serum silver concentration of 55 ppb in an adult human of normal blood volume.

In U.S. Pat. No. 5,985,301, Nakamura describe cellulose fiber that contains silver as an antibacterial agent. In short, cellulose is dissolved in a particular type of solvent, and then silver compounds are added. Fibers are then spun from these solutions. It is reported that these fibers have bactericidal properties. This method must be considered a drug-releasing technology. This fact is emphasized by the following quote from the Nakamura patent: “ . . . enhancing antibacterial effects presumably by promoting the discharge of silver ions from the silver-based antibacterial agent.”

It is known that certain quaternary ammonium salts possess antimicrobial properties. Examples include benzethonium chloride and benzalkonium chloride (BACTINE). It is also known that certain low molecular weight quaternary ammonium groups can be incorporated into polymeric substrates (without chemical bonding) in order to provide certain degrees of antimicrobial activity.

Ionene polymers or polymeric quaternary ammonium compounds (polyquats), i.e., cationic polymers containing quaternary nitrogens in the polymer backbone, belong to a well-known class of biologically-active compounds. See, e.g., A. Rembaum, Biological Activity of lonene Polymers, Applied Polymer Symposium No. 22, 299-317 (1973). Ionene polymers have a variety of uses in aqueous systems such as microbicides, bactericides, algicides, sanitizers, and disinfectants. U.S. Pat. Nos. 3,778,476, 3,874,870, 3,898,336, 3,931,319, 4,013,507, 4,027,020, 4,089,977, 4,111,679, 4,506,081, 4,581,058, 4,778,813, 4,970,211, 5,051,124, and 5,093,078 give various examples of these polymers, their preparation, and their uses. U.S. Pat. Nos. 3,778,476, 3,898,536, and 4,960,590, in particular, describe insoluble tri-halide containing ionene polymers. U.S. Pat. No. 4,013,507 describes ionene polymers which selectively inhibit the growth of malignant cells in vitro.

Hou et al., U.S. Pat. No. 4,791,063, teach polyionene-transformed modified polymer-polysaccharide separation matrices for use in removing contaminants of microorganism origin from biological liquids. This patent teaches that absorption of bacterial cells by ion-exchange resins is attributable to electrostatic attraction between quaternary ammonium groups on the resin surface and carboxyl groups on the bacteria cell surface.

Chen et al. describe the preparation of antimicrobial dendrimers (highly-branched polymers) having quaternary ammonium functionality (Chen, et al., “Quaternary Ammonium Functionalized Poly(propylene imine) Dendrimers as Effective Antimicrobials: Structure-Activity Studies”, Biomacromolecules 1 p473-480 (2000)). The compounds described therein are soluble in water; hence, they are not suitable for use as wound dressing materials. The enhanced antimicrobial properties exhibited by polymeric quaternary compounds (relative to monomeric quats), is discussed therein, and also in several other references (Ikeda, T., “Antibacterial Activity of Polycationic Biocides”, Chapter 42, page 743 in: High Performance Biomaterials, M. Szycher, ed., Technomic, Lancaster Pa., (1991); Donaruma, L. G., et al., “Anionic Polymeric Drugs”, John Wiley & Son, New York, (1978); Ikeda T, Yamaguchi H, and Tazuke, S “New Polymeric Biocides: Synthesis and Antibacterial Activities of Polycations with Pendant Biguanide Groups”; Antimicrob. Agents Chemother. 26(2), p139-44 (1984)).

U.S. Pat. No. 6,039,940, to Perrault, teaches a composition and method for treating a wound with an inherently antimicrobial dressing based on quaternary ammonium polymers. The dressing is a hydrogel containing, by weight, about 15 to 95 percent, and preferably about 61 to 90 percent, and most preferably about 65 to 75 percent, cationic quaternary amine acrylate polymer. This polymer is prepared by the polymerization of acryloyloxyethyl(or propyl)-trialkyl(or aryl)-substituted ammonium salts or acrylamidoethyl(or propyl)-trialkyl(or aryl)-substituted ammonium salts. Ultraviolet light is used as the catalyst. The antimicrobial hydrogels are stated to be non-irritating to the wound, absorb wound exudate, and, due to the inherently antimicrobial properties, “enhance the sterile environment around the wound.”

However, the human trial detailed in the '940 disclosure concluded that the measured parameter, the ‘irritation potential’, was “not significantly different from the control”. Analysis of the numerical results indicates that no hydrogel dressing tested gave a better improvement during the test period than the control. This suggests that the use of a hydrogel, although providing a “soothing effect” according to the '940 patent, may not be the optimum dressing for wound healing. Also, it is believed that the present invention offers an advantage over dressing such as the '940 dressing in that the dressing of the present invention are applied to non-gel dressings (e.g., they are dry rather than hydrated). This provides a greater potential for uptake of wound exudates and other aqueous solutions, compared to the '940 hydrogels, which already have water occupying sites in the composition.

Also, the polymers described in the '940 patent are based on acrylate or acrylamide, and as such are susceptible to hydrolysis. Hydrolysis is expected to be greater at high pH or at low pH. It should be noted that these materials are hydrogels, and thus contain a significant amount of water (e.g., 5% to 85%). It also states that the hydrogels are preferably prepared with a physical support structure in order to better retain the hydrogel over a wound. Although the possibility of forming these hydrogels around a web or fibril support is given, it is not clear that the hydrogel material is bonded to the support in any manner. The materials can be dried to powders and later reconstituted. The fact that the hydrogel materials are powders when dry would indicate that shedding of loose hydrogel particles is to be expected. A device wherein the absorbent material is permanently bound to a structural substrate would be more desirable.

The hydrogel materials described by the '940 patent are not breathable. That is, they do not permit unrestricted passage of air through the samples. In that method, the spaces between strands of the “support material” are essentially clogged by hydrated hydrogel material. In the current invention, the individual filaments and fibrils within the substrate are separately “coated” with covalently-bonded antimicrobial polymer. Note that this can be achieved even on a basic raw material such as cotton lint, or wood pulp, which can then be formed into a fabric or other useful structure. Such a process is not possible with the technology described by the '940 patent. In that case, the non-bonded composite must be formed over a prefabricated structure which is a time-consuming process.

The '940 patent also teaches that residual monomer concentrations of up to 3% are acceptable. Such a high level of residual monomer would undoubtedly result in release of active antimicrobial agent into a wound. Monomers such as those used to produce the hydrogels described in the '940 patent are known to cause skin and eye irritation, as well as sensitization (MSDS # 14491: Ciba Specialty Chemicals Corporation). While not being bound to a particular theory, another source of leaching of antimicrobial activity may be poor or incomplete cross linking; this may be a source of leaching in that the polymers are only linked to one another, rather to a substrate. It is also difficult and time-consuming to completely wash a hydrogel in order to extract all residual monomer and other leachables without destroying the network structure. The leaching may become greater or less based on ambient conditions of wound exudate. Evidence of leaching from the hydrogel composition are in the '940 patent's summary of the Kirby-Bauer zone of inhibition data. That data, presented in Table 1 of the '940 patent, demonstrate large zones of inhibition, up to 20 mm, around a 5 mm square hydrogel sample. While the '940 patentees meant this data to indicate the antimicrobial effectiveness of the hydrogel, a proper interpretation, in comparison with the non-leaching attributes of the present invention, indicates that the '940 compositions demonstrate a drug-releasing/leaching phenomenon.

In conclusion with regard to the above descriptions of the art, it is apparent that there is a need for an improved dressing that has an effective antimicrobial coating or layer on it, which is covalently bound to a substrate, and is non-leaching upon use.

The antimicrobial activity of a polystyrene fiber containing covalently bonded tertiary amine groups was tested by Endo et al. (“Antimicrobial Activity of Tertiary Amine Covalently Bonded to a Polystyrene Fiber”, Applied Journal of Environmental Microbiology 53(9), p2050 (1987). Only very slight antimicrobial activity was found for these fibers in the absence of other agents. Significant antimicrobial activity was only observed when these fibers were combined with other antimicrobial agents such as deoxycholate or actinomycin (leachable antibiotics). The material described is based on polystyrene, and thus it is not expected to have physical properties suitable for an absorbent wound dressing material.

The cerium (IV) ion initiated graft polymerization of vinyl monomers onto hydroxyl-containing substrates was first described by Mino (G. Mino and S. Kaizerman; “A New Method for the Preparation of Graft Copolymers. Polymerization Initiated by Ceric Ion Redox Systems”, Journal of Polymer Science 31(22), p242 (1958)). The mechanism and kinetics of Ce(IV)-initiated graft polymerization of vinylacetate-acrylonitrile onto PVA in water solution was studied by Odian and Kho (G. Odian and J. H. T. Kho; “Ceric Ion Initiated Graft Polymerization onto Poly(vinyl Alcohol)”, J. Macromolecular Science—Chemistry A4(2) p317-330, (1970)). Later, Vitta et al. described the grafting of methacrylic acid onto solid cellulose substrates (S. B. Vitta, et al., “The Preparation and Properties of Acrylic and Methacrylic Acid Grafted Cellulose Prepared by Ceric Ion Initiation. Part I. Preparation of the Grafted Cellulose”, J. Macromolecular Science—Chemistry A22(5-7) p579-590 (1985)). None of these references describe antimicrobial materials, or graft polymers based on quaternary ammonium compounds.

A large number of other inventors have labored to produce germicidal bandage and packing materials. U.S. Pat. No. 5,441,742 to Autant et al. discloses a modified cellulosic material with biocidal properties. Unfortunately, water releases the biocidal agents from the material with the concomitant problems of irritation or toxicity towards surrounding tissues. Iodine has been a favored biocidal material. Both U.S. Pat. No. 5,302,392 to Karakelle et al. and U.S. Pat. No. 5,236,703 to Usala rely on polymers containing polyvinylpyrollidone to bind and release iodine. Another approach is shown in U.S. Pat. No. 5,091,102 to Sheridan which relies on the presence of a cationic surfactant to provide germicidal properties to a dry fabric. All of these inventions suffer the problem of having a more or less toxic germicide that can leach from the material.

We have recently identified additional unexpected applications of the quaternary amine polymer chemistry defined herein. Such novel applications include the binding, whether through covalent linkages, ionic interactions, adsorption, or other mechanisms, of significant levels of quaternary amine polymers to powder substrates, including but not limited to mica. By the term “powder” for purposes of this invention, what is meant is monodisperse to polydisperse compositions of particle sizes ranging from the sub-micron (very fine) to millimeter size particles, depending on the relevant application. Mica is a commonly used component of cosmetics applied to the skin. Accordingly, significant application of this technology to the cosmetic arts and other arts in which it is desirable to limit microbe proliferation and viability through use of antimicrobial powders. Additional applications of this technology include, for example, treatment of athlete's foot, (Tinia pedis), jock itch (Tinea cruris), chaffing, and other dermatological conditions in which opportunistic infections or irritations need to be controlled.

Furthermore, through high level grafting of quaternary amine polymers according to this disclosure, the properties of powders (including but not limited to micas), fabrics and other substrates may be modified to increase the capacity and firmness of dye molecule binding to the cationically treated substrates of this invention. In addition, according to another embodiment of this invention, a wide variety of ionic biologically active molecules may be bonded to polyionic substrates prepared according to various aspects or modifications of this invention. Thus, with respect to wound dressings, a wide variety of antibiotics, proteins, peptides, matrix metalloproteinase inhibitors, analgesics, anti-inflammatory compounds, and the like are exhibit net anionic charge at physiological pH, or pH's encountered at a wound site. By contacting these anions with polyquaternary amine functionalized substrates, prepared according to the methods of the present disclosure, the association of these anionic compounds with the substrate is stabilized. Through mass action, displacement of ions and similar mechanisms, the anions associated with the polycationic substrate of this invention are released over time, to exhibit desirable biological effects over a more extended period than would be the case if the biologically active compound were merely absorbed or adsorbed in, on or to a substantially ionically neutral substrate. Thus, in one embodiment of the present invention, anionic antibiotics are associated with a polycationic wound dressing. Over time, the anionic antibiotic is released into the wound and is associated with the wound dressing, thereby achieving the double benefits of keeping the wound dressing free of microbial contamination, and extended release of antibiotic into the otherwise at-risk of infection wound site. In another embodiment of the present invention, anionic matrix metalloproteinase inhibitors, MMPI, (e.g. the carboxylic acid form of Ilomastat) is associated with a polycationic wound dressing for bed sores, ulcers and the like. Over time, the MMPI is released into the wound to reduce or prevent ulcer or bed sores from forming, from getting worse, and to induce healing thereof. In the non-wound-care arena, the principles of this embodiment of the invention may be extended into release over time of ionic biologically active compounds from polyionically treated clothing substrates, to reduce or eliminate body odor. Thus, ionic fragrance molecules may be associated with the polyionic substrate of this invention, and released over time, or, antimicrobial compounds may be released from the polyionic substrate to eliminate or reduce bacterial activity at the skin surface, which is considered generally as the mode by which non-pungent sweat components are converted to pungent microbial metabolic products. Based on this disclosure, those skilled in the art will appreciate that a wide variety of applications may be considered. Thus, for example, yet another application of this invention is in the field of water, air or other fluid filtration, whereby ionic biologically active compounds are associated with a polyionic filter medium, which then over time releases the biologically active compounds into the fluid medium as it passes through the filtration material.

All patents, patent applications and publications discussed or cited herein are understood to be incorporated by reference to the same extent as if each individual publication or patent application was specifically and individually set forth in its entirety.

From the above review, it is apparent that what is needed in the art of would dressings is a broad spectrum antibacterial or antimicrobial agent that remains in the bandage material where it can prevent bacterial growth, without exerting any negative effects on adjacent living tissue.

Similarly, a need also exists for other products to have non-leaching, antibacterial or antimicrobial surfaces to act prophylactically to prevent or reduce the presence of pathogens on such surfaces. Specific examples of such applications of the present technology are provided in the following section. Further, the present invention provides methods and compositions for extended association of and controlled over time release of ionic biologically active compounds from select substrates.

### SUMMARY OF THE INVENTION

The present invention provides methods and compositions for an antimicrobial and/or antibacterial composition comprising a substrate over which a non-leaching polymeric coating is covalently bonded. The polymeric coating contains a multitude of quaternary ammonium groups which exert activity against microbes, and also is absorptive of aqueous solutions. A preferred method of fabrication is also described.

One object of the present invention is to make a wound dressing that comprises an absorbent, non-leaching antimicrobial surface over a suitable dressing substrate. A typical substrate is cellulose, rayon, or other fibrous mesh, such as a gauze pad. Surprisingly, tests have proved that certain novel forms of polymers on a wound dressing substrate, while not rising to the definition of “superabsorbent” in the parent application, are in fact highly effective at reducing or eliminating the numbers of microbes, including fungi and viruses in addition to a range of bacteria. These embodiments have been shown to be non-leaching. It has also been shown that such materials can be produced on a variety of substrates without significant changes in the physical properties of the substrates such as texture, color, odor, softness, or mechanical strength.

Another object of the present invention is to provide a superabsorbent polymer material having antibacterial properties.

Another object and embodiment of the present invention demonstrates the effectiveness of such a superabsorbent polymer.

Another object of the present invention is a composition that comprises a substrate with a covalently bonded superabsorbent polymer surface having antimicrobial properties, which is covered or surrounded by a second substrate to which is covalently bonded another layer of polymer which is not superabsorbent, but which does have a high level of antimicrobial groups. This combination provides both high capacity of liquids absorption, and a high level of antimicrobial activity, while maintaining the feel and handling characteristics of a conventional fabric, particularly in the preferred configuration in which the outermost layer is the non-superabsorbent polymer described herein.

Another object of the invention is the inclusion in a dressing or pad according to the present invention of an indicator that indicates a condition or the status of the recipient based on some aspect of fluid or other input from the user. For instance, an indicator (such as a color indicator) on a wound dressing may indicate the type of infection or the presence of HIV antibodies, and an indicator (such as a color indicator) on a tampon or like pad may indicate whether or not the user is pregnant, or her HIV status.

Another object of the present invention is to provide methods and compositions that pertain to antimicrobial surfaces for a variety of supplies and equipment, including a sanitary pad, a tampon, a diaper, a sponge, a sanitary wipe, food preparation surfaces, and other surfaces in need of a non-leaching antimicrobial property.

An additional object is to provide, via binding, whether through covalent linkages, ionic interactions, adsorption, or other mechanisms, of significant levels of quaternary amine polymers to powder substrates, including but not limited to mica for inclusion in cosmetic, antifungal, and like compositions whether in a dry or moist form.

A further object of this invention is to provide a method for treatment of athlete's foot, (Tinia pedis), jock itch (Tinea cruris), chaffing, and other dermatological conditions in which opportunistic infections or irritations need to be controlled.

In yet a further embodiment of this invention, it is an object, through high level grafting of quaternary amine polymers according to this disclosure, to modify the properties of powders (including but not limited to micas), fabrics and other substrates to increase the capacity and avidity of dye molecule binding to the cationically treated substrates of this invention.

A further object of this invention is to provide compositions and methods whereby a wide variety of biologically and even chemically active compounds are associated with and then released from a polyionic substrate to achieve extended retention and release characteristics in a wide variety of applications from appropriately selected substrates.

### DETAILED DESCRIPTION OF THE INVENTION

Definitions:

For the purposes of this disclosure, certain definitions are provided. By “non-hydrolyzable” is meant a bond that does not hydrolyze under standard conditions to which a bond is expected to be exposed under normal usage of the material or surface having such bond. For instance, in a wound dressing according to the present invention that has “non-hydrolyzable” bonds, such “non-hydrolyzable” bonds do not hydrolyze (e.g., undergo a hydrolysis-type reaction that results in the fission of such bond) under: normal storage conditions of such dressing; exposure to would exudates and/or body fluids when in use (e.g., under exposure to an expected range of pH, osmolality, exposure to microbes and their enzymes, and so forth, and added antiseptic salves, creams, ointments, etc.). The ranges of such standard conditions are known to those of ordinary skill in the art, and/or can be determined by routine testing.

By “non-leaching” is meant that sections of the polymer of the present invention do not appreciably separate from the material and enter a wound or otherwise become non-integral with the material under standard uses. By “not appreciably separate” is meant that no more than an insubstantial amount of material separates, for example less than one percent, preferably less than 0.1 percent, more preferably less than 0.01 percent, and even more preferably less than 0.001 percent of the total quantity of polymer. Alternately, depending on the application, “not appreciably separate” may mean that no adverse effect on wound healing or the health of an adjacent tissue of interest is measurable.

In regard to the above, it is noted that “non-leachable” refers to the bond between the polymer chain and the substrate. In certain embodiments of the present invention, a bond between the polymer backbone and one or more type of antimicrobial group may be intentionally made to be more susceptible to release, and therefore more leachable. This may provide a benefit where it is desirable for a percentage of the antimicrobial groups to be selectively released under certain conditions. However, it is noted that the typical bond between the polymer chain and antimicrobial groups envisioned and enabled herein are covalent bonds that do not leach under standard exposure conditions.

Polymers according to the present invention have the capacity to absorb aqueous liquids such as biological fluids (which are defined to include a liquid having living or dead biologically formed matter, and to include bodily fluids such as blood, urine, menses, etc.). The capacity to absorb an aqueous liquid can be measured by the grams of water uptake per gram of absorbent material in a single instance. One general definition for a superabsorbent polymer is that such polymer generally would be capable of absorbing, in a single instance, about 30 to 60 grams of water per gram of polymer. A broader definition could include polymers that absorb less than 30 grams of water per gram of polymer, but that nonetheless have enhanced capacity to absorb water compared to similar materials without such enhanced capacity. Alternately, an “absorbent” as opposed to a “superabsorbent” polymer may be defined as a polymer that has a capacity to absorb aqueous liquids, but which normally will not absorb over 30 times its weight in such liquids.

By “degree of polymerization” is meant the number of monomers that are joined in a single polymer chain. For example, in a preferred embodiment of the invention, the average degree of polymerization is in the range of about 5 to 1,000. In another embodiment, the preferred average degree of polymerization is in the range of about 10 to 500, and in yet another embodiment, the preferred average degree of polymerization is in the range of about 10 to 100.

A substrate is defined as a woven or nonwoven, solid, or flexible mass of material upon which the polymers of the invention can be applied and with which such polymers can form covalent bonds. Cellulose products, such as the gauze and other absorbent dressings described in the following paragraphs, are preferred materials to be used as water-insoluble bases when a wound dressing is prepared. The term “substrate” can also include the surfaces of large objects, such as cutting boards, food preparation tables and equipment, surgical room equipment, floor mats, blood transfer storage containers, cast liners, splints, air filters for autos, planes or HVAC systems, military protective garments, face masks, devices for protection against biohazards and biological warfare agents, lumber, meat packaging material, paper currency, powders, including but not limited to mica for cosmetic, antifungal or other applications, and other surfaces in need of a non-leaching antimicrobial property, and the like, onto which is applied the antimicrobial polymeric coating in accordance with the present invention. Apart from cellulose, any material (ceramic, metal, or polymer) with hydroxyl groups or reactive carbon atoms on it's surface can be used as a substrate for the cerium (IV) or other free radical, redox or otherwise catalyzed grafting reaction described in the following paragraphs. The extent of grafting will be dependent on the concentration of surface hydroxyl groups and the concentration of available reactive carbons. Even materials which do not normally contain sufficient surface hydroxyl groups may be used as substrates, as many methods are available for introducing surface hydroxyl groups. These methods generally include hydrolysis or oxidation effected by methods such as heat, plasma-discharge, e-beam, UV, or gamma irradiation, peroxides, acids, ozonolysis, or other methods. It should be noted that methods other than cerium initiated grafting may also be used in the practice of this invention. Thus, for example, not meant to be limiting, a free radical initiator may be used to initiate monomer polymerization. So-called “Azo” initiators, such as VA-057, V-50 and the like, available from Wako Pure Chemical Industries, may be utilized. Other initiators, including but not limited to hydrogen peroxide, sodium persulfate (“SPS”), and the like may also be utilized to advantage according to this invention to initiate polymerization.

The term NIMBUS™ is a coined term used herein as an acronym to refer to a substrate according to the present invention which to refer to Novel Intrinsically Microbicidal Utility Substrates whereby a substrate is derivatized to exhibit antimicrobial efficacy. Thus, polyquaternary amine derivatized substrates exhibit this property. Likewise, a polyquaternary amine derivatized substrate which has been charged with anionic antibiotic, likewise exhibits this property.

Various materials were investigated by the inventors as substrates for the preparation of absorbent dressings containing covalently-bonded, polymeric quaternary ammonium biocidal agent. Among these materials were several commercially-available gauze and surgical sponge products, including several materials manufactured by Johnson & Johnson Company (J&J). J&J's, “NU GAUZE”, General use sponge (referred to in this application as “sub#1”), J&J's “STERILE GAUZE Mirasorb sponge” (herein referred to as “sub#4”), and J&J's “SOFT WICK” dressing sponge (herein referred to as “sub#5”) were all used to prepare working prototypes. All three materials are rayon/cellulose (sub #4 also contains polyester) sheets with non-woven mesh-like structures, and a fiber surface area much greater than traditional woven cotton-fiber gauze. Sub#1 and sub#4 are a single 8″×8″ sheet which is folded into a 4-layer sheet measuring 4′×4′, and both weigh approximately 1.45 to 1.50 grams per sheet. Sub#5 has a denser structure, and is made from a single 12″×8″ sheet folded into a 6-layer sheet measuring 4″×4′, weighing approximately 2.5 grams.

In addition, several types of fabric materials were also used as substrates, including: “Fruit of the Loom” 100% cotton knitted tee-shirt material, “Gerber” 100% cotton bird's-eye weave cloth diaper material, “Cannon” 100% cotton terry wash-cloth material, “Magna” yellow, non-woven wiping cloth (75% rayon, 25% polyester), and “Whirl” cellulose kitchen sponge”; referred to herein as: “subTS”, “subDIA”, “subWC”, “subMag”, and “subCKS” respectively. The scope of this invention is not limited to the use of materials mentioned herein as substrates.

Modification of these substrates to prepare absorbent materials with antimicrobial properties was achieved by immersing the substrates into aqueous solutions of vinyl monomers containing quaternary ammonium groups. Reaction of these monomers with the substrate materials to form graft polymers was catalyzed by ceric ion (Ce⁺⁴), Azo initiators, SPS, or peroxide. A typical modification procedure is detailed in Example 1. Other samples were prepared according to the same basic procedure; however, different substrates, monomers, reaction conditions, washing/drying procedures were used. This data is summarized in Table 1.

Additionally, another aspect of the present invention is the inclusion in a dressing of a hemostatic agent. Hemostatic compounds such as are known to those skilled in the art may be applied to the dressing, either by bonding or preferably added as a separate component that dissolves in blood or wound exudates, and acts to reduce or stop bleeding. In addition, the high positive charge density conferred on substrates due to the application of quaternary amine polymers according to this invention itself provides a surface which facilitates the coagulation cascade.

Finally, a substrate is defined as a woven or nonwoven, solid, or flexible mass of material upon which the polymers of the invention can be applied and with which such polymers can form covalent bonds. Cellulose products, such as the gauze and other flexible absorbent dressings described in the following paragraphs, are preferred materials to be used as flexible substrates when a wound dressing is prepared. The term “substrate” can also include the surfaces of large, generally non-flexible objects, such as cutting boards, food preparation tables and equipment, and surgical room equipment, and other large flexible or generally non-flexible objects such as a floor mats, a blood transfer storage containers, cast liners, splints, air filters for autos, planes or HVAC systems, military protective garments, face masks, devices for protection against biohazards and biological warfare agents, lumber, meat packaging materials, paper currency, powders including but not limited to mica, and other surfaces in need of a non-leaching antimicrobial property, and the like, onto which is applied the antimicrobial polymeric coating in accordance with the present invention. Apart from cellulose, any material (ceramic, metal, or polymer) with hydroxyl groups or available reactive carbons on it's surface can be used as a substrate for the cerium (IV) and other initiator catalyzed grafting reactions described in the following paragraphs. The extent of grafting will be dependent on the surface hydroxyl concentration and the concentration of susceptible carbon atoms. Even materials which do not normally contain sufficient surface hydroxyl groups may be used as substrates, as many methods are available for introducing surface hydroxyl groups. These methods generally include hydrolysis or oxidation effected by methods such as heat, plasma-discharge, e-beam, UV, or gamma irradiation, peroxides, acids, ozonolysis, or other methods. It should be noted that methods other than cerium initiated grafting may also be used in the practice of this invention.

Furthermore, it will be noted based on the present disclosure that antimicrobial applications of surface treated mica have wide applicability to cosmetics, in which mica is an almost universally included component, with or without titanium dioxide treatment. Inclusion of mica treated according the present disclosure provides a solution, for example, to the situation where a mascara applicator is used, returned to a reservoir bearing adherent microbes which, in the absence of the antimicrobial mica, proliferate in the reservoir. Such proliferation has given rise to increasing levels of concern in the industry and this invention provides a novel, significant and unexpected solution to this long felt need. In addition, the increased dye-binding affinity of substrates, including mica, treated according to the present invention, has applicability to the fabric and cosmetic arts.

The use of cerium(IV) salts as graft polymerization initiators is described above. These salts function by a redox mechanism involving complex formation between the metal ion and the hydroxyl groups on the cellulose substrate. It is known that other metal ions such as V(V), Cr(VI), and Mn(III) function in a similar manner (see P. Nayak and S. Lenka, “Redox Polymerization by Metal Ions ”, J. Macromolecular Science, Reviews in Macromolecular Chemistry, C19(1), p83-134 (1980).

Persulfate ion is known as a water-soluble initiator for vinyl polymerizations, but is not widely recognized as a catalyst for graft polymerizations. We have found that sodium persulfate (SPS) functions as a grafting catalyst much in the same manner as the cerium salts used in the parent application (see Examples 3-8, below). There is an advantage for materials prepared from this new catalyst vs. materials prepared using cerium salts, in that the finished materials prepared using SPS show zero discoloration. Samples prepared using cerium catalysts may show a slight off-white, or yellowish discoloration under certain conditions. For most consumer applications it is desirable to have a pure white product. It is possible that materials prepared using the cerium catalyst can contain a small amount of residual cerium, which might be undesirable in the finished product. This is not the case for the SPS system. The by-products of the SPS catalyst are simply sodium ion and sulfate ion, which are completely safe and nontoxic. In general, it is not desirable to have any heavy metal residues in finished medical devices, since some of the heavy metal catalysts described in the above paragraph are rather toxic (chromium, for instance), and could pose hazards for personnel involved in manufacturing, as well as pollution and environmental concerns. An additional benefit of the SPS catalyst is that polymerization may be carried out at room temperature, if desired (see example #4). The grafting reaction using SPS also appears to be quicker than the cerium salt catalyzed reaction. Significant grafting can be achieved in 30 minutes at 60° C. (see example #5), and presumably even quicker at higher temperatures.

The use of peroxydiphosphate and peroxydisulfate as initiators for the graft polymerization of vinyl monomers (but not quaternary monomers) onto silk and wool fibers has been described (see M. Mishra, Graft Copolymerization of Vinyl Monomers onto Silk Fibers, J. Macromolecular Science, Reviews in Macromolecular Chemistry C19(2), p193-220 (1980). These systems often rely on redox pairs formed by the oxidants (peroxydisulfate or peroxydiphosphate) with reductants such as lithium bromide, or silver nitrate, or are done in the presence of acids such as H₂SO₄. Again, the use of metals such as silver and lithium may lead to undesirable residues in the final products. The use of strong acids is unsuitable for the grafting of cellulose substrates due to severe substrate damage.

Banker reports, in U.S. Pat. No. 5,580,974, the preparation of microfibrillated oxycellulose suitable for use as a carrier in agricultural, cosmetic, and topical and transdermal drug products, and as a binder and disintegrant in the making of tablets, prepared by the oxidation of cellulosic materials with persulfate salts in water, with or without the presence of an aqueous inorganic acid, or in glacial or aqueous acetic acid. No mention of graft polymerization is made.

We have also found (unexpectedly) that other compounds are also capable of catalyzing the grafting of quaternary vinyl monomers onto cellulose. Hydrogen peroxide (HP) is an effective catalyst for this reaction (see Example 9). It is surprising that HP functions in this manner. Although peroxides are generally known to be capable of initiating vinyl polymerizations, it is also well known that oxygen interferes with these processes. Reaction of HP with organic materials liberates elemental oxygen, but this apparently did not prevent grafting. HP is rather useful in that it may be the cleanest catalyst available for preparing these types of graft copolymers. The by-products of HP-catalyzed copolymerization are simply water and oxygen.

The by-product of SPS-catalyzed copolymerization is sulfate ion. Sulfate ion is not toxic; however, it is conceivable that its presence in some systems may be undesirable. The HP-catalyzed materials are also very white, with zero discoloration.

Azo compounds such as AIBN (2,2′-azobisisobutyronitrile) are commonly used as initiators for vinyl polymerizations, but are not generally thought of as catalysts for preparation of graft copolymers. We have found, however, that a water-soluble derivative of AIBN (2,2′-Azobis[N-(2-carboxyethyl)-2-methylpropionarnidine]tetrahydrate, or VA-057, available from Wako Specialty Chemicals) was a suitable initiator for the graft polymerization of quaternary vinyl monomers onto cellulose (see Example 10). AIBN, which is one of the most commonly used polymerization initiators, is not soluble in water; and thus cannot be used directly in aqueous solutions, as can the various compounds described above. AIBN is soluble in alcohols, however, and thus can possibly be used as an initiator for the graft polymerization of quaternary monomers onto cellulose since the monomers are also soluble in alcohols. It is also likely that AIBN could be used in an emulsion system in order to achieve similar results. Other potentially useful Azo initiators include: (2,2′-Azobis[2-(5-methyl-2-imidazolin-2-yl)propane]dihydrochloride, or VA-041; 2,2′-Azobis{2-methyl-N-[1,1-bis(hydroxymethyl)-2-hydroxyethyl]propionamide, or VA-080; 2,2′-Azobis(2-methylpropionamide)dihydrochloride, or V-50; 2,2′-Azobis(N-cyclohexyl-2-methylpropionamide), or Vam-111; 1,1′-Azobis(cyclohexane-1-carbonitrile); all available from Wako Specialty Chemicals, Inc.; and numerous other similar compounds).

Organic peroxides such as benzoyl peroxide (BPO) are also widely used as polymerization initiators. Just as in the case of AIBN (above), BPO is not water soluble, but it can possibly be used in alcoholic solution in order to graft quaternary vinyl monomers onto cellulose. Other potentially useful peroxide initiators include: (dicumyl peroxide, t-butyl peroxide, methylethylketone peroxide, and a variety of other peroxides, peroxyketals, peroxydicarbonates, and hydroperoxides). These and numerous other potentially useful catalysts are available from a variety of suppliers such as Lucidol-Penwalt, and Akzo.

Combinations of two or more of the initiators described above are also effective (see Example 11). These catalysts can also be used to form crosslinked cellulose-quatemary grafted materials (see example #12).

It should also be noted that the mechanism of action of quaternary compounds is directed towards the cell membrane of the target organism. This process has been described as a mechanical “stabbing” (on a molecular level) which causes rupture of the cell membrane. Thus, it is not possible for pathogenic organisms to develop resistance as observed for most antibiotics.

A further embodiment according to this invention, comprises a wound dressing material capable of controlled or sustained release of a drug such as an antibiotic. However, based on the present disclosure, those skilled in the art will appreciate that the method and compositions disclosed in connection with this embodiment of the invention are not limited to antimicrobials. A variety of other agents, including, for example, matrix metalloproteinase inhibitors, MMPI's, such as Illomostat and its ionic derivatives, may be associated with and released from select polyionic substrates according to this disclosre. Likewise for vitamins, dyes, or other active chemicals such as fragrances. Accordingly, applications of this aspect of the invention are not limited to wound dressings, and include a wide range of applications as specified herein. It should further be noted that the controlled release function of substrates according to this aspect of the invention is in addition to the good antimicrobial properties of polyquatemary amine functionalized substrates as disclosed herein.

In a further aspect according to this embodiment of the invention, an appropriately polyionically derivatized substrate according to this invention is sold as a device, and is loaded with a drug, fragrance, or any of a wide variety of different ionic compounds at the point of sale or use by qualified personnel.

Those skilled in the art will appreciate that many drugs are negatively charged (such as penicillin or vitamin C, as sodium ascorbate). These negatively charged drugs form an ionic bond with a polyquatemary amine derivatized substrate, and prevent them from being washed out quickly from the thus derivatized substrate following ionic interaction between the drug and the polycationic substrate. In comparison, simply coating or infusing a normal untreated substrate (such as cotton or rayon) with drug allows it to be more quickly leached or washed out from the substrate. Complexes formed between the polycationic substrate of this invention and different compounds will have different binding constants, and thus the rate of release will be different. This can be controlled by adjusting the amount of positive charge (graft level), by adjusting the level of drug loading, or by controlling other factors such as surface area or pH. The concept can be extended to positively charged drugs simply by using a negatively charged, i.e. polyanionically derivatized substrate. This is done by grafting acrylic acid monomer onto cellulose, for instance.

In addition to biologically active compounds which are classically considered to be “drugs”, compositions and methods according to this invention can also bind and release more simple ionic compounds such as metal ions (calcium, zinc, silver, rubidium, etc.). Some of these ions are known to be important in wound healing (see, for example, U.S. Pat. No. 6,149,947, hereby incorporated by reference for this purpose). Alternatively, for example, hypochlorite ion may be associated with the derivatized substrate of this invention, and released as an antimicrobial, both for medical or non-medical applications. In yet other embodiments according to this invention, sodium pyrithione is used as a drug to treat fungal skin infections (athlete's foot and dandruff), thereby yielding clothing applications (e.g. socks, undershirts, underwear, derivatized with a polyquatemary ammonium loaded with antifungally effective amounts of sodium pyrithione), foot powder (powder, e.g. “talc” treated with the polyquatemary ammonium polymer according to this invention, and loaded with an antifungally effective amount of sodium pyrithione or another appropriate antifungal). We have demonstrated the enhanced retention of sodium pyrithione (SP) and release over different washing cylces of antimicrobially effective amounts of SP, as compared with complete wash-out in a single cycle of SP using standard substrates.

Illomostat, other MMPIs, and other wound care agents may likewise be associated with and released from the polyionic substrate according to this invention. Both GM1489 molecule (which has a carboxylic acid rather than the hydroxamic acid at the N-terminus of Ilomastat), and the C-terminal carboxylic acid form of Ilomastat (rather than the N-methyl amide in Ilomastat) have a negative charge at physiological pH. Thus, both MMP inhibitors are expected to reversibly bind to NIMBUS, much as do indicator dye molecules and negatively charged antibiotic molecules, as exemplified herein below. Accordingly, wound dressings according to this invention provide sustained release of these potent MMPI molecules. GM1489 has Ki values for MMPs that are almost as good as Ilomastat, and while the Ki values for the C-terminal carboxylic form of Ilomastat are lower, it is still a very acceptable and potent MMPI.

Further, polycationic substrate according to this invention provides sustained release of “PHI or polyhydrated ionogen” active ingredient in Greystone Medical's DerMax dressing. Likewise for proteins such as serine protease inhibitor, alpha-1 protease inhibitor and gelatin (denatured collagen) since these proteins exhibit negative charges at pH 7. Accordingly, per this disclosure, a substrate according to this invention charged with these biologically active compounds provides a dressing with the ability to inhibit MMPs and serine proteases, as is the case for Promogran dressing, except that such a dressing according to this invention would be expected to have better performance for ulcers and bed sores and other wounds caused or exacerbated by matrix metalloproteinases and serine proteinases, because it binds and releases over time inhibitors for both classes of proteases.

In yet a further embodiment of the present invention, a gel, hydrogel, or SAP is utilized as a component of this aspect of the invention. In particular, SAP-polyquatemary ammonium derivatized substrate according to this invention exhibits significant additional advantages. In addition, while grafted polyquatemary amine derviatized substrates are a preferred mode of this invention, simply coated, or otherwise immobilized polyquaternary amine treated substrates are likewise anticipated to operate according to the principles disclosed herein for grafted substrates.

In a yet further embodiment according to this invention, interpenetrating networks (IPNs), or IPNs combined with covalent bonding, is utilized as a variation within the scope of the present invention. Accordingly, coatings are made from polyquat copolymers. For example, a copolymer of TMMC and MMA, soluble in alcohol, but insoluble in water, is permeable or swellable in water. Such a composition is applied from alcohol solution, and does not wash off in water even though it is not covalently bonded. Such a substrate is then charged with polyanionic compounds with desired chemical or biological activities for binding to and then sustrained release from the substrate.

### EXAMPLE 1

### Production of Absorbent Anti-Microbial Compounds

A commercially available surgical sponge rayon/cellulose gauze material (sub#4) was unfolded from its as-received state to give a single layer sheet measuring approximately 8″ by 8″. The sample was then refolded “accordion-style” to give a 6-layer sample measuring approximately 1.33″ by 8″. This was then folded in the same manner to give a 24-layer sample measuring approximately 1.33″ by 2″. This refolding was done so as to provide uniform and maximum surface contact between the substrate and reaction medium, in a small reaction vessel. A solution was prepared by mixing 0.4 grams of ammonium cerium (IV) nitrate (CAN) (Acros Chemical Co. cat # 201441000), 25.0 mL [2-(methacryloyloxy)ethyl]trimethylammonium chloride (TMMC) (Aldrich Chemical Company, cat# 40,810-7), and 55 mL of distilled water. This solution was placed into a 250 mL wide-mouth glass container equipped with a screw-cap lid, and argon gas was bubbled vigorously through the solution for 60 seconds. The folded gauze substrate was placed into the solution, and the solution was again sparged with argon for 30 seconds. The container was capped while being flushed with a stream of argon gas. The container was placed into an oven set at 75° C., and gently agitated by hand every 30 minutes for the first two hours, then every hour for the next 4 hours. After a total of 18 hours, the jar was removed from the oven and allowed to cool to room temperature. The sample was removed from the jar, unfolded, and thoroughly washed three times with water, being allowed to soak in water for at least 30 minutes between washings. These sequential washings, also termed rinsings, remove effectively all of the non-polymerized monomer molecules, non-stabilized polymer molecules, and catalyst, such that the final composition is found to not leach its antimicrobial molecules, by routine detection means known and used by those of ordinary skill in the art. By non-stabilized polymer molecules is meant any polymer molecule that has neither formed a covalent bond directly with a binding site of the substrate, nor formed at least one covalent bond with a polymer chain that is covalently bonded (directly or via other polymer chain(s)) to the substrate.

After these rinsings, excess water was removed from the sample by gently squeezing. Further dewatering was accomplished by soaking the sample in 70% isopropanol for 30 minutes. Excess alcohol was removed by gently squeezing the sample, which was then allowed to dry overnight on a paper towel in open air. The sample was then dried in vacuum at room temperature for 18 hours. The sample was allowed to stand in air for 15 minutes before being weighed. The final weight of the sample was measured to be 2.13 grams. The initial weight of the sample before treatment was 1.45 grams. The percent of grafted polymer in the final product was calculated as follows: (2.13-1.45)/2.13×100=31.9%. Some disruption of the fiber packing of the mesh was observed, and this resulted in a “fluffier” texture for the treated material.

Preparations of additional samples were performed according to similar procedures using substrates, antimicrobials and reaction conditions. The reaction conditions and percent ata for each sample are summarized in Table 1.

TABLE 1
Ceric ion initiated grafting of gauze substrates

| | | | [Monomer] | | | | | |
|---|---|---|---|---|---|---|---|---|
| Sample# | Substrate | Monomer | (mol/L) | [Ce+](mM) | T (° C.) | Total Vol. | % Grafting | |
| 1 | #4 | TMMC | 1 | 11 | 75 | 80 mL | 12% | |
| 2 | #1 | TMMC | 1.2 | 14 | 75 | 80 mL | 34% | |
| 3 | #1 (×2) | TMMC | 1.2 | 14 | 75 | 80 mL | 32% | |
| 4 | #1 | TMMC | 1.2 | 9 | 75 | 80 mL | 32% | |
| 5 | #1 (×2) | TMMC | 1.2 | 9 | 75 | 80 mL | 20% | |
| 6 | #1 | TMMC | 0.7 | 14 | 75 | 80 mL | 28% | |
| 7 | #1 (×2) | TMMC | 0.7 | 14 | 75 | 80 mL | 27% | |
| 8 | #1 | TMMC | 1 | 11 | 75 | 80 mL | 37% | |
| 9 | #1 | TMMC | 1 | 11 | 75 | 80 mL | 37% | |
| 10 | #1 | TMAC | 1.2 | 11 | 75 | 80 mL | 25% | |
| 11 | #1 | TMAPMC | 1.2 | 10 | 75 | 90 mL | <1% | |
| 12 | #1 | TMAS | 0.9 | 11 | 75 | 80 mL | 20% | |
| 13 | #1 | DADMAC | 1.4 | 10 | 75 | 90 mL | 6% | |
| 14 | #4 (×2) | TMMC | 1.3 | 15 | 90 | 60 mL | degraded | |
| 15 | #4 (×2) | TMMC | 0.5 | 11 | 90 | 85 mL | 5% | |
| 16 | #4 (×2) | TMMC | 0.7 | 15 | 90 | 60 mL | 13% | |
| 17 | #4 | TMMC | 0.7 | 15 | 90 | 60 mL | 9% | |
| 18 | #1 | TMMC | 1.3 | 15 | 90 | 60 mL | degraded | |
| 19 | #1 | TMMC | 0.7 | 15 | 90 | 60 mL | 23% | |
| 20 | #1 | TMMC | 0.4 | 15 | 90 | 60 mL | 17% | |
| 21 | #4 | TMMC | 1.3 | 15 | 90 | 60 mL | 26% | |
| 22 | #4 | TMMC | 0.7 | 8 | 75 | 60 mL | 7% | |
| 23 | #1 | TMMC | 2 | 20 | 75 | 60 mL | 30% | |
| 24 | #1 | TMMC | 2 | 5 | 75 | 60 mL | <1% | |
| 25 | #1 | TMMC | 0.7 | 20 | 75 | 60 mL | 25% | |
| 26 | #1 | TMMC | 0.7 | 7 | 75 | 60 mL | 15% | |
| 27 | #1 | TMAS | 0.4 | 7 | 60 | 200 mL | 14% | |
| 28 | #1 | TMAS | 0.2 | 2 | 60 | 200 mL | 11% | |
| 29 | #1 | TMAS | 0.8 | 10 | 60 | 200 mL | 19% | |
| 30 | #1 | TMMC | 1 | 11 | 50 | 80 mL | 44% | |
| 31 | #5 | TMMC | 1 | 11 | 50 | 80 mL | 48% | |
| 32 | #5 | TMMC | 1 | 11 | 50 | 80 mL | 48% | |
| 33 | #5 | VBTAC | 0.7 | 78 | 60 | 35 mL | 15% | |
| 34 | #5 | DADMAC | 2 | 60 | 60 | 60 mL | 7% | |
| 35 | #5 | VBTAC | 0.4 | 50 | 60 | 37 mL | 20% | |
| 36 | DIA | TMMC | 0.8 | 11 | 50 | 150 mL | 12% | |
| 37 | WC | TMMC | 1 | 18 | 65 | 200 mL | 22% | |
| 38 | MAG | TMMC | 1 | 18 | 65 | 100 mL | 39% | |
| 39 | CKS | TMMC | 0.8 | 11 | 60 | 150 mL | 11% | |
| 40 | TS | TMMC | 1 | 15 | 50 | 150 | 17% | |
| 41 | #5 | TMMC/ | 0.7 | 15 | 60 | 122 mL | 64% | |
| | | SR344 | 2.00% | | | | | |
| 42 | #5 | TMMC/ | 0.4 | 15 | 60 | 224 mL | 79% | |
| | | SR344 | 2.00% | | | | | |
| 43 | #5 | TMMC | 0.9 | 10 | 75 | 85 mL | 18% | 30 min. |
| 44 | #5 | TMMC | 0.9 | 10 | 80 | 85 mL | 21% | 15 min. |
| 45 | #5(×2) | TMMC | 0.9 | 10 | 55 | 170 mL | 32% | 2 hours |

NOTES for Table 1:
TMMC = [2-(Methacryloyloxy)ethyl]trimethylammonium chloride (75% solution in water) Aldrich Chemical #40,810-7
TMAS = [2-(Acryloyloxy)ethyl]trimethylammonium methyl sulfate (80% solution in water) Aldrich Chemical #40,811-5
TMAC = [2-(Acryloyloxy)ethyl]trimethylammonium chloride (80% solution in water) Aldrich Chemical #49,614-6
TMAPMC = [3-(Methacryloylamino)propyl]trimethylammonium chloride (50% solution in water) Aldrich Chemical #28,065-8
VBTAC = vinylbenzyltrimethylammonium chloride Acros Chemical #42256
DADMAC = diallyldimethylammonium chloride (65% solution in water) Aldrich Chemical #34,827-9
SR344 = poly(ethylene glycol)diacrylate Sartomer Company # SR344

All procedures were performed in 500 mL or 250 mL screw-cap glass jars overnight (approximately 18 hours), except for samples #43-45 which were reacted for indicated times.

The samples prepared as shown in Table 1 indicated that high-yield grafting of vinyl monomers containing quaternary ammonium groups onto various textile substrate materials can be achieved under rather mild conditions. The appearance of the prepared biocidal absorbent dressings generally was identical to that of the starting material. Parameters such as mechanical strength, color, softness, and texture were found to be sufficient and acceptable for use in the various applications mentioned above. For instance, the materials based on medical dressings were soft, white, odorless, and absorbent. Storage of these materials for several months yielded no observable physical changes. The same holds true for heat treatments of 75° C. for several hours (this is not meant to be a limiting condition).

It should be noted that although these examples demonstrate modification of textile fabrics already in finished form, it is also within the scope of this invention to achieve the grafting modification at the raw materials stage. Threads, yams, filaments, lints, pulps, as well as other raw forms may be modified and then fabricated into useful materials or fabrics (woven or nonwoven) by weaving, knitting, spinning, or other forming methods such as, spunbonding, melt blowing, laminations thereof, hydroentanglement, wet or dry forming and bonding, etc.

Grafting yields were found to be reproducible with constant formulation and reaction conditions. Samples were thoroughly washed to remove any residues such as unreacted monomer or homopolymer. Degree of grafting was calculated based on the weight of the starting material and the final dried weight of the grafted material. The calculated values of percent grafting are subject to a certain degree of error based upon the fact that the materials appear to contain a small amount of adsorbed water due to exposure to the laboratory atmosphere. This is true even for the untreated starting materials which were generally found to show a reversible weight loss of approximately 5 to 7% after being dried in a 60° C. oven for 30 minutes. Another potential source of error is the possibility of the presence of other counterions besides chloride (bromide, or nitrate, for instance). Experiments were conducted to correlate the weight of treated samples after washing with excess salt solutions of various composition. Related to this is the well-known observation that quaternary ammonium compounds strongly bind sodium fluorescein dye to form a colored complex. Various samples from Table 1 were tested by immersing them in a concentrated (5%) solution of sodium flourescein, followed by drying, and then thorough washing in water. Untreated fabrics did not retain any color after this treatment; however, all treated materials showed a pronounced color which ranged from light orange to dark brown, depending on the quaternary ammonium content. In one case (a sample identical to that of Sample #31), the fluorescein treated sample showed a weight gain of 27%. Further analysis on this sample for % nitrogen and % chloride was conducted by an independent laboratory (Galbraith Laboratories, Inc., Knoxville, Tenn.). The results (2.62% N and 6.83% Cl) indicate a slightly lower level than as calculated gravimetrically. This is likely due to the reasons described above. An exact control of % grafting is not a requirement of this invention. As described in the testing presented below, the antimicrobial activity of these materials is fuctional over a wide range of compositions.

The materials described by Sample #1 through Sample #40 are graft copolymers in which the quaternary ammonium polymeric grafts have a linear structure. These highly charged linear chains would be water-soluble if they were not tethered at one end to a cellulose substrate. Thus, the materials are capable of absorbing and holding water. Selected materials were tested for their ability to absorb and retain water. For instance, a 2.22 gram sample of the material of Sample #2 was found to retain 12.68 times its own weight of water when placed in a funnel and completely saturated. The samples prepared in Sample #41 and Sample #42 were found to retain water at 38 and 66 times their own weight, respectively. These two samples were prepared using a combination of monofunctional quaternary monomer, and a difunctional non-quatemary cross linking agent. The cross linking agent causes the grafted polymer chains to become branched, and also allows individual chains to form chemical bonds with each other that result in network formation. Once swollen with water, the polymer network becomes a slippery gel material. The absorbent biocidal materials produced with and without cross linking agent have similar chemical and antimicrobial properties. Although the materials prepared using cross linking agents have extremely high absorbing capacity, they do tend to become rather slippery when wet.

This slippery property may be undesirable in some applications, particularly where this is the exposed surface. However, the two different variations may be utilized in conjunction with each other. For instance, the material of Sample #35 may be used as a shell or barrier material around the material of Sample #42. This would result in a bandage material having a superabsorbent compound interiorly to provide absorptive capacity, having inherent antimicrobial properties throughout, and having superior antimicrobial properties on the exterior (where a polymer having antimicrobial properties that are demonstrated superior to a polymer with superabsorptive capacity is employed in the outer location).

### EXAMPLE 2

### Testing of Antimicrobial Activity

All biological testing was performed by an independent testing laboratory (Biological Consulting Services of North Florida, Incorporated, Gainesville, Fla.). The first set of antimicrobial activity tests was performed using the absorbent antimicrobial material of Sample # 21. The grafting yield for this sample was 26%. An untreated, unwashed sample of as-received sub#4 was used as a control. A sample of sub#1 treated with a siloxane based quaternary formulation (TMS, or 3-(trimethoxysilyl)-propyloctadecyldimethyl ammonium chloride) was also tested (sample # 1122F). This sample contained approximately 9% quaternary siloxane which was applied from methanol solution. Based on a series of experiments with this quaternary siloxane, this is the maximum level which could be successfully applied to the substrate material. It was later found that the applied siloxane quaternary treatment was unstable, as evidenced by significant weight loss after washing the treated material after 30 days storage. This level is also higher than is typically achieved in antimicrobial treatments of similar substrates using commercial TMS products. It should also be noted that there were difficulties during the testing due to the hydrophobic (water-repellent) nature of the siloxane-treated material. Such properties are not desirable in a product designed specifically to be highly absorbent.

In a modification of the AATCC-100 antimicrobial test protocol, gauze material from these three samples was aseptically cut into squares weighing 0.1±0.05 grams. This corresponds to a 1′×1″ four-layer section. Each square was then individually placed in a sterile 15-mm petri dish and covered. One-milliliter tryptic soy broth suspension containing 10⁶-cfu/ml mid-log phase E. coli (ATCC 15597) or S. aureus (ATCC 12600) was added to each gauze section. The plates were then incubated overnight at 37° C. Following incubation, the material was aseptically placed into 50-mL conical centrifuge tubes. Twenty-five milliliters of sterile phosphate buffered saline was then added to each tube. The tubes were shaken on a rotary shaker (Red Rotor PR70/75, Hoofer Scientific, CA) for 30 minutes. The eluant was then diluted accordingly and enumerated by aseptically spread plating onto Tryptic Soy Agar (TSA) plates. The plates were incubated overnight at 37° C. All gauze samples were processed in triplicates. The results of this testing are summarized in Table 2.

TABLE 2
Results of antimicrobial activity testing.

| | cfu/mL | |
|---|---|---|
| Sample | Staphylococcus aureus | Escherichia coli |
| Sub#4 (control) | 1.3 × 10⁶ | 6.1 × 10⁶ |
| | 4.6 × 10⁵ | 2.4 × 10⁶ |
| | 8.0 × 10⁵ | 1.5 × 10⁶ |
| Material of Sample #21 | <10 | <10 |
| | <10 | <10 |
| | <10 | <10 |
| TMS siloxane Material | <10 | 1.4 × 10⁴ |
| | 20 | 2.3 × 10⁴ |
| | 170 | 4.3 × 10⁴ |

The results of this experiment are rather self-explanatory, and indicate that the material of Sample #21 was able to kill greater than 99.999% of both organisms. The siloxane-based quaternary ammonium sample (DC5700) was fairly effective on S. aureus, but only slightly effective on E. coli.

Further testing was carried out using the materials of Sample # 9. A freshly-prepared sample of sub#1 treated with TMS siloxane quaternary ammonium (8%) was also tested, along with a washed untreated sub#1 control. In this experiment, freshly-prepared bacterial cultures containing additional TSB growth medium were used. The samples were treated as before. In addition, a second set of samples was reinoculated with additional bacterial culture after the first day of incubation, and allowed to incubate for an additional day. Data from these experiments is presented in Tables 3 and 4.

TABLE 3
Colony forming units (cfu) of 4 layer gauze strips cut into one
inch² sections following inoculation with
bacteria and overnight incubation.

| | cfu/mL | |
|---|---|---|
| Sample | Staphylococcus aureus | Escherichia coli |
| (Control) | 5.2 × 10⁷ | 8.7 × 10⁷ |
| (Sub#1 washed) | 2.1 × 10⁷ | 4.6 × 10⁷ |
| | 9.4 × 10⁷ | 5.4 × 10⁷ |
| TMS siloxane quat | 1.2 × 10⁶ | 8.8 × 10⁶ |
| (8% on Sub#1) | 9.1 × 10⁶ | 1.3 × 10⁷ |
| | 5.9 × 10⁶ | 7.0 × 10⁶ |
| Material of Sample #9) | 8.9 × 10¹ | 6.6 × 10¹ |
| (37% TMMC on sub#1) | 3.7 × 10¹ | 3.6 × 10¹ |
| | 3.3 × 10¹ | 9.0 × 10⁰ |

TABLE 4
Colony forming units (cfu) of 0.1-gram gauze strips following
inoculation with the indicated bacteria, overnight incubation,
re-inoculation, and overnight incubation.

| | cfu/mL | |
|---|---|---|
| Sample | Staphylococcus aureus | Escherichia coli |
| (Control) | 5.6 × 10⁸ | 3.9 × 10⁸ |
| (Sub#1 washed) | 2.6 × 10⁸ | 3.8 × 10⁸ |
| | 4.2 × 10⁸ | 1.9 × 10⁸ |
| TMS siloxane quat | 2.1 × 10⁶ | 2.2 × 10⁸ |
| (8% on Sub#1) | 1.8 × 10⁶ | 1.8 × 10⁸ |
| | 8.0 × 10⁵ | 2.7 × 10⁸ |
| Material of Sample #9 | 3.4 × 10¹ | 6.7 × 10² |
| (37% TMMC on sub#1) | 3.8 × 10² | 7.2 × 10¹ |
| | 9.1 × 10¹ | 5.9 × 10¹ |

Again, the results of this experiment are self-explanatory. The siloxane-based quaternary ammonium did not show significant antibacterial activity, whereas the TMMC-grafted material did.

In another experiment, the antimicrobial effectiveness of several materials was tested in the presence of a high concentration of bodily fluids, as expected to occur in a heavily draining wound, for instance. The procedure was similar to that described above, except that the bacterial levels were higher (10⁸ cfu/mL), and the inoculation mixture contained 50/50 newborn calf serum and TSB. The samples tested in this experiment were those of Samples #30 and 31. In addition, a sample of siloxane quaternary ammonium-treated knitted cotton material was obtained from a commercial supplier (Aegis). The results are presented in Table 5.

TABLE 5
Testing of biocidal
absorbent materials in presence of 50% calf blood serum

| | cfu/mL | |
|---|---|---|
| Sample | Staphylococcus aureus | Escherichia coli |
| Control | 5.9 × 10⁷ | 2.7 × 10⁷ |
| “Sub#5” | 6.3 × 10⁷ | 1.9 × 10⁷ |
| J&J gauze | 7.1 × 10⁷ | 9.8 × 10⁶ |
| Siloxane quat on | 1.8 × 10⁷ | 1.2 × 10⁶ |
| Cotton fabric | 3.5 × 10⁷ | 9.5 × 10⁵ |
| | 1.5 × 10⁷ | 7.0 × 10⁶ |
| Material of Sample 30 | 1.0 × 10⁴ | <1.0 × 10⁰ |
| TMMC quat | 1.2 × 10⁴ | 5.0 × 10⁰ |
| ˜44% graft | 9.7 × 10³ | <1.0 × 10⁰ |
| Material of Sample 31 | 2.4 × 10⁴ | 3.9 × 10² |
| TMMC quat | 3.2 × 10⁴ | 6.0 × 10⁰ |
| ˜48% graft | 1.2 × 10⁵ | 1.0 × 10⁰ |

As can be seen from the data in Table 5, the siloxane based quaternary treated material showed almost zero effectiveness. The TMMC-grafted material was extremely effective against e-coli, even in the presence of high concentrations of bodily fluids. The high serum protein concentration appeared to mask the effectiveness of the TMMC-grafted material to some extent; however, the levels of serum which were used in this experiment were quite challenging. Generally, in these types of experiments much lower serum levels are used (10 to 20%).

It is an aim of this invention to provide an absorbent antimicrobial material which does not leach or elute any soluble antimicrobial agent. In order to verify this, material of sample #31 (Table 1) was extraction tested under a range of pH conditions, and also in the presence of blood serum. In addition, a commercially available antimicrobial dressing was also tested under identical conditions. The commercially available antimicrobial dressing is “Kerlix-A.M.D. Antimicrobial Super Sponges”, manufactured by Kendall Tyco Healthcare Group (active ingredient 0.2% Polyhexamethylene Biguanide). The following procedure was used: Approximately, a one square inch section of each bandage material was placed in a 50-mL sterile polypropylene tube. Twenty-five milliliters of phosphate buffered saline at pH 5.0, pH 7.0, pH 7.0 supplemented with 10% fetal bovine serum (FBS), or pH 9.0 was added to each tube. Each sample was processed in triplicates to assure reproducibility. pH values were adjusted using 0.1 N NaOH or HCl. The tubes ware then placed on a rotary shaker (Red Rotor PR70/75, Hoofer Scientific, CA) for and agitated mildly (40 rotations/min) for 16 hours. Tryptic Soy Agar (TSA) (Difco Laboratory, Detroit, Mich.) petri dishes were inoculated with a continuous lawn of either E. coli (ATCC 15597) or S. aureus (ATCC 12600) and the plates were divided into four sections. Twenty microliters of the soaked gauze aqueous extract was then placed onto the labeled sections of the bacteria inoculated plates. The plates were then covered and incubated at 37° C. for 18 hours. The plates were then visibly inspected for growth suppression at areas of inoculation. The results are presented in Table 6.

TABLE 6
Anti Microbial release test of supplied gauze material after
soaking in Phosphate buffered saline (PBS) for
16 hours at various pH values

| | Effect of Gauze Extract on Bacterial Growth | |
|---|---|---|
| Sample | Staphylococcus aureus | Escherichia coli |
| pH 5.0 | No Inhibition | No Inhibition |
| Material of Sample #31 | No Inhibition | No Inhibition |
| | No Inhibition | No Inhibition |
| pH 7.0 | No Inhibition | No Inhibition |
| Material of Sample #31 | No Inhibition | No Inhibition |
| | No Inhibition | No Inhibition |
| PH 7.0 | No Inhibition | No Inhibition |
| Material of Sample #31 | No Inhibition | No Inhibition |
| with 10% FBS | No Inhibition | No Inhibition |
| PH 9.0 | No Inhibition | No Inhibition |
| Material of Sample #31 | No Inhibition | No Inhibition |
| | No Inhibition | No Inhibition |
| PH 5.0 | Inhibition | No Inhibition |
| (Kerlix AMD) | Inhibition | No Inhibition |
| | Inhibition | No Inhibition |
| PH 7.0 | Inhibition | No Inhibition |
| (Kerlix AMD) | Inhibition | No Inhibition |
| | Inhibition | No Inhibition |
| PH 7.0 | Inhibition | No Inhibition |
| With 10% FBS | Inhibition | No Inhibition |
| (Kerlix AMD) | Inhibition | No Inhibition |
| PH 9.0 | Inhibition | No Inhibition |
| (Kerlix AMD) | Inhibition | No Inhibition |
| | Inhibition | No Inhibition |

As seen from the results listed in Table 6, the material of sample #31 did not leach or release any antimicrobial agent under any of the conditions tested; however, the commercial antimicrobial dressing, Kerlix AMD, was found to release antimicrobial agent toxic to S. aureus under all testing conditions. Such leaching of active agent may have an undesirable effect on wound healing, and also cause decreased antimicrobial effectiveness of the dressing.

Further antimicrobial testing in the presence of 10% blood serun was performed using additional organisms as described below. These included a number of common pathogenic bacteria, as well as at least one fungal species. The material of Sample # 32 was tested, and untreated sub#5 was used as a control. The gauze material was aseptically cut into approximately one inch square sections. Sub#5 gauze sample consisted of material in four layers and the material of Sample #32 consisted of two layers. Both types of samples weighed approximately 0.1 gram. Each sample section was individually placed in a sterile 100×15-mm petri dish and covered. Escherichia coli (ATCC 15597), Staphylococcus aureus (ATCC 12600), Klebsiella pneumoniae (ATCC 13883), Pseudomonas aeruginosa (ATCC 51447), Proteus vulgaris (ATCC 13115), Serratia marcescens (ATCC13880), Enterococcus faecalis (ATCC 19433), and Enterobacter aerogenes (ATCC 13048) were grown in twenty five milliliters of tryptic soy broth (TSB) (Difco Laboratory, Detroit, Mich.) for 16 hours at 37° C. Each bacterial culture was then diluted in Fresh TSB or PBS containing 10% Fetal Bovine Serum (Sigma, St. Louis, Mo.) to a final concentration of approximately 10⁵-cfu/mL. One milliliter of each bacterial suspension was added to each gauze section. Each section was inoculated with only one bacterial species. All gauze samples were inoculated in triplicates. The petri dish containing the inoculated sample was then incubated for 18 hours at 37° C. in 95% humidity. Following incubation, the gauze material was aseptically placed into 50-mL conical centrifuge tubes. Twenty-five milliliters of sterile phosphate buffered saline (PBS) was then added to each tube. The tubes were shaken on a rotary shaker (Red Rotor PR70/75, Hoofer Scientific, CA) for 30 minutes. The eluant was then serially diluted. Tenfold dilutions were performed by the addition of 0.3-ML of sample to 2.7-mL of sterile PBS. Aliquots of each dilution or of the original undiluted sample were then aseptically spread plated onto Tryptic Soy Agar (TSA) (Difco Laboratory, Detroit, Mich.) plates. The plates were incubated for 18 hours at 37° C. The colonies on the respective plates were counted and concentrations were determined. The fungus Candida albicans was used in the same procedure outlined above, except incubation times were doubled and Sabouraud Dextrose Broth and agar (Difco Laboratory, Detroit, Mich.) were used instead of TSB and TSA, respectively. The results are summarized in Table 7. The reported bacterial levels are diluted by a factor of 25× versus the level present in the actual gauze samples.

TABLE 7
Antimicrobial activity results for various organisms.

Sample

| Organism | Sub 5* (control) | Material of Sample 32 |
|---|---|---|
| S. aureus | 5.9 × 10⁶ | <2.0 × 10⁰ |
| S. aureus | 6.3 × 10⁷ | <2.0 × 10⁰ |
| S. aureus | 7.1 × 10⁷ | <2.0 × 10⁰ |
| E. coli | 1.7 × 10⁶ | <2.0 × 10⁰ |
| E. coli | 1.9 × 10⁶ | <2.0 × 10⁰ |
| E. coli | 2.4 × 10⁶ | <2.0 × 10⁰ |
| K. pneumoniae | 1.8 × 10⁶ | <2.0 × 10⁰ |
| K. pneumoniae | 1.4 × 10⁶ | <2.0 × 10⁰ |
| K. pneumoniae | 3.7 × 10⁶ | <2.0 × 10⁰ |
| P. aeruginosa | 2.1 × 10⁷ | <2.0 × 10⁰ |
| P. aeruginosa | 3.9 × 10⁷ | <2.0 × 10⁰ |
| P. aeruginosa | 4.3 × 10⁷ | <2.0 × 10⁰ |
| P. vulgaris | 2.8 × 10⁶ | <2.0 × 10⁰ |
| P. vulgaris | 1.1 × 10⁷ | <2.0 × 10⁰ |
| P. vulgaris | 3.7 × 10⁶ | <2.0 × 10⁰ |
| S. marcescens | 6.7 × 10⁷ | <2.0 × 10⁰ |
| S. marcescens | 7.3 × 10⁷ | <2.0 × 10⁰ |
| S. marcescens | 8.7 × 10⁷ | <2.0 × 10⁰ |
| E. faecalis | 3.8 × 10⁶ | <2.0 × 10⁰ |
| E. faecalis | 1.7 × 10⁶ | <2.0 × 10⁰ |
| E. faecalis | 2.9 × 10⁶ | <2.0 × 10⁰ |
| E. aerogenes | 1.1 × 10⁷ | <2.0 × 10⁰ |
| E. aerogenes | 3.3 × 10⁷ | <2.0 × 10⁰ |
| E. aerogenes | 2.9 × 10⁷ | <2.0 × 10⁰ |
| C. albicans | 5.9 × 10⁵ | 2.0 × 10⁰ |
| C. albicans | 7.2 × 10⁵ | 4.0 × 10⁰ |
| C. albicans | 1.2 × 10⁶ | 5.0 × 10⁰ |

*values represent cfu/mL of the 25-mL PBS solution used to elute the microorganisms from the gauze sections.

The results presented in Table 7 indicate significant antimicrobial activity for the TMMC-grafted material against a variety of organisms. Further testing of this material was conducted using several bacteriophages. Bacteriophages are viral organisms which infect a particular bacterial host. In this method, the antimicrobial material is inoculated with the viral agent and then allowed to incubate for a specified period. The amount of viable viral organism is then determined on the basis of remaining ability to infect the host bacteria. Samples were aseptically cut into approximately one inch² square sections. Sub#5 gauze sample consisted of material in four layers and the material of Sample #32 consisted of material in two layers. Each sample weighed approximately 0.1 g. Each sample section was individually placed in a sterile 100×15-mm petri dish and covered. Stocks of the following bacteriophages, MS2 (ATCC 15597-B1), φX-174 (ATCC 13706-B1), and PRD-1 were added to 10 mL of TSB or PBS containing 10% Fetal Bovine Serum (Sigma, St. Louis, Mo.) to a final concentration of approximately 10⁶-cfu/mL. One milliliter of each bacterial suspension was added to each gauze section. All gauze samples were inoculated in triplicates. The petri dish containing the inoculated sample was then incubated for 18 hours at 37° C. in 100% humidity. Following incubation, the gauze material was aseptically placed into 50-mL conical centrifuge tubes. Twenty-five milliliters of sterile phosphate buffered saline (PBS) was then added to each tube. The tubes were shaken on a rotary shaker (Red Rotor PR70/75, Hoofer Scientific, CA) for 30 minutes. The eluant was then serially diluted. Tenfold dilutions were performed by the addition of 0.3-ML of sample to 2.7-mL of sterile PBS. Phages were assayed as plaque-forming units (pfu) using their respective hosts (MS2 (ATCC 15597-B1), Escherichia coli C-3000 (ATCC 15597); φX-174 (ATCC 13706-B1), E. coli (ATCC 13706); and PRD-1, Salmonella typhimurium (ATCC 19585)). The soft-agar overlay method (Snustad, S. A. and D. S. Dean, 1971, “Genetic Experiments with Bacterial Viruses”. W. H. Freeman and Co., San Francisco) was used for enumerating the phages. The results are presented in Table 8.

TABLE 8
Testing of absorbent antimicrobial material against viral agents.

Sample

| Bacteriophage | Sub #5 (control)* | Material of Sample #32 |
|---|---|---|
| MS-2 | 3.3 × 10⁴ | <2.0 × 10⁰ |
| MS-2 | 4.1 × 10⁴ | <2.0 × 10⁰ |
| MS-2 | 2.3 × 10⁴ | <2.0 × 10⁰ |
| PRD1 | 1.7 × 10⁵ | 1.2 × 10² |
| PRD1 | 7.9 × 10⁴ | 1.5 × 10² |
| PRD1 | 8.8 × 10⁴ | 1.7 × 10² |
| φX-174 | 8.7 × 10³ | 2.4 × 10³ |
| φX-174 | 1.2 × 10⁴ | 1.1 × 10³ |
| φX-174 | 9.0 × 10³ | 1.7 × 10³ |

*values represent pfu/mL of the 25-mL PBS solution used to elute the microorganisms from the gauze sections.

As seen from the results in Table 8, the absorbent antimicrobial material has significant effectiveness against viral pathogens, as evidenced by reduction or loss of bacteriophage activity in the treated sample #32. These results, in combination with the results regarding bacterial and fungal organisms, indicate a relatively broad antimicrobial potential for compositions of the invention.

In additional testing, several absorbent antimicrobial dressing materials not based on acrylate materials were studied. These tests included the materials of Samples #33, with VBTAC, and #34, with DADMAC. The material was aseptically cut into two layer square sections of approximately one inch². Each square was individually placed in a sterile 100×15-mm petri dish and covered. E. coli (ATCC 15597) and S. aureus (ATCC 12600) were grown in twenty five milliliters of tryptic soy broth (TSB) (Difco Laboratory, Detroit, Mich.) for 16 hours at 37° C. Each bacterial culture was then diluted in Fresh TSB or PBS containing 10% Fetal Bovine Serum (Sigma, St. Louis, Mo.) to a final concentration of approximately 10⁵-cfu/mL. One milliliter of each bacterial suspension was added to each gauze section. Each section was inoculated with only one bacterial species. All gauze samples were inoculated in triplicates. The petri dish containing the inoculated sample was then incubated for 16 hours at 37° C. in 95% humidity. Following incubation, the gauze material was aseptically placed into 50-mL conical centrifuge tubes. Twenty-five milliliters of sterile phosphate buffered saline (PBS) was then added to each tube. The tubes were shaken on a rotary shaker (Red Rotor PR70/75, Hoofer Scientific, CA) for 30 minutes. The eluant was then serially diluted. Tenfold dilutions were performed by the addition of 0.3-ML of sample to 2.7-mL of sterile PBS. Aliquots of each dilution or of the original undiluted sample were then aseptically spread plated onto Tryptic Soy Agar (TSA) (Difco Laboratory, Detroit, Mich.) plates. The plates were incubated for 18 hours at 37° C. The colonies on the respective plates were counted and concentrations were determined. The results are summarized in Table 9.

TABLE 9
Colony forming units (cfu) present in the PBS eluant (25-mL) of
the indicated gauze sections (1-inch²) following their
inoculation with bacteria and overnight incubation:

| | cfu/mL of the PBS eluant | |
|---|---|---|
| Sample | Staphylococcus aureus | Escherichia coli |
| SUB 5 | 7.6 × 10⁶ | 1.6 × 10⁷ |
| (CONTROL) | 6.9 × 10⁶ | 2.9 × 10⁷ |
| | 5.8 × 10⁶ | 1.3 × 10⁷ |
| Material of Sample #33 | <1.0 × 10⁰ | <1.0 × 10⁰ |
| 15% VBTAC graft | <1.0 × 10⁰ | <1.0 × 10⁰ |
| | <1.0 × 10⁰ | <1.0 × 10⁰ |
| Material of Sample #34 | 3.0 × 10⁰ | <1.0 × 10⁰ |
| 7% DADMAC graft | 4.0 × 10⁰ | <1.0 × 10⁰ |
| | <1.0 × 10⁰ | <1.0 × 10⁰ |

As shown in the table, the material with grafted quaternary ammonium polymer showed significant antimicrobial activity, even in the presence of 10% blood serum.

Additional verification of the nonleaching nature of the subject materials was obtained by Kirby-Bauer zone of inhibition tests. Sample #32 was used in this experiment, along with a control of substrate #5. The following procedure was used: Material was aseptically cut into: 0.5×0.5 cm square sections, 0.2×2.0 cm strips, and 1.0×6.0 strips. All material was used in one-layer sections. Escherichia coli (ATCC 15597), and Staphylococcus aureus (ATCC 12600) were grown in five milliliters of tryptic soy broth (TSB) (Difco Laboratory, Detroit, Mich.) for 5 hours at 37° C. 0.5-mL of either bacterial culture was then added to molten (45° C.) sterile Tryptic Soy Agar (TSA) (Difco Laboratory, Detroit, Mich.). The mixture was then swirled and poured into a 15×100-mm petri dish. The gauze material was then aseptically placed onto the surface of the agar and the agar was allowed to solidify. The petri dish containing the sample was then incubated for 18 hours at 37° C. in 95% humidity. Zones of bacterial growth inhibition were then measured. Results are shown in Table 10.

TABLE 10
Results of zone of inhibition testing of Sample #32

Zone of inhibition
around sample (mm)

| Sample/section size | S. aureus | E. coli |
|---|---|---|
| SUB 5/1.5 × 1.5 | <0.1 | <0.1 |
| SUB 5/2.0 × 2.0 | <0.1 | <0.1 |
| SUB 5/1.0 × 5.0 | <0.1 | <0.1 |
| Sample #32/1.5 × 1.5 | <0.1 | <0.1 |
| Sample #32/2.0 × 2.0 | <0.1 | <0.1 |
| Sample #32/1.0 × 5.0 | <0.1 | <0.1 |

As shown in Table 10, no measurable zone of inhibition was observed around either the control or treated samples.

A study was conducted to determine the speed of antimicrobial action for the subject materials. Material similar in composition to sample #33 (code # 0712A) was used in this study, along with an untreated control (substrate #5). The following procedure was employed. Material was aseptically cut into approximately one inch square sections. 0712A gauze sample consisted of material in two layers, and SUB-5 samples were in 3 layers. Each sample section was individually placed in a sterile 100×15-mm petri dish and covered. Staphylococcus aureus (ATCC 12600) was grown in twenty-five milliliters of tryptic soy broth (TSB) (Difco Laboratory, Detroit, Mich.) for 6 hours at 37° C. The bacterial culture was then diluted in Fresh 1% TSB or 1×PBS containing 10% Fetal Bovine Serum (Sigma, St. Louis, Mo.) to a final concentration of approximately 10⁶-cfu/mL. One half (0.5) milliliter of the bacterial suspension was added to each gauze section. All gauze samples were inoculated in duplicates. The petri dish containing the inoculated sample was then incubated for the indicated time points at 37° C. in 95% humidity. Following incubation, the gauze material was aseptically placed into 50-mL conical centrifuge tubes. Twenty-five milliliters of sterile phosphate buffered saline (PBS) was then added to each tube. The tubes were shaken on a rotary shaker (Red Rotor PR70/75, Hoofer Scientific, CA) for 10 minutes. The eluant was then serially diluted. Tenfold dilutions were performed by the addition of 0.3-ML of sample to 2.7-mL of sterile PBS. Aliquots of each dilution or of the original undiluted sample were then aseptically spread plated in duplicates onto Tryptic Soy Agar (TSA) (Difco Laboratory, Detroit, Mich.) plates. The plates were incubated for 18 hours at 37° C. The colonies on the respective plates were counted and concentrations were determined. Results of this rate study are presented in Table 11.

TABLE 11
Effect of 0712A and Sub 5 gauze material on the inactivation of
Staphylococcus aureus at different exposure times.

Sample (and respective
bacterial count at
specified times)

| Time | Sub 5 | 0712A |
|---|---|---|
| 1 minute | 1.5 × 10⁵ | 3.0 × 10² |
| | 1.9 × 10⁵ | 3.1 × 10² |
| 10 minutes | 1.3 × 10⁵ | 2.0 × 10² |
| | 2.5 × 10⁵ | 8.0 × 10¹ |
| 20 minutes | 1.5 × 10⁵ | 8.0 × 10¹ |
| | 2.3 × 10⁵ | 1.2 × 10² |
| 30 minutes | 1.6 × 10⁵ | 1.1 × 10¹ |
| | 2.8 × 10⁵ | 2.1 × 10¹ |
| 60 minutes | 1.9 × 10⁵ | 1.2 × 10¹ |
| | 2.1 × 10⁵ | 1.9 × 10¹ |
| 4 hours | 3.3 × 10⁵ | 3 × 10⁰ |
| | 2.5 × 10⁵ | 1.2 × 10¹ |
| 8 hours | 4.0 × 10⁶ | <2.0 × 10⁰ |
| | 4.8 × 10⁶ | 4.0 × 10⁰ |
| 12 hours | 2.3 × 10⁷ | 6.0 × 10⁰ |

¹values represent cfu/mL of the 25-mL PBS solution used to elute the microorganisms from the gauze sections.

The data clearly indicates that significant antimicrobial activity is manifested very quickly. Approximately 99.8% of S. aureus is destroyed in as little as one minute.

Samples similar in composition to that of sample #31 in Table 1 were subjected to sterilization by several methods including: autoclaving, ethylene oxide exposure, gamma irradiation (2.5 Mrad), and electron beam irradiation (2.5 Mrad). No observable degradation of physical properties or loss of antimicrobial activity was observed.

Samples #43, #44 and #45 (see Table 1) were reacted for significantly shorter periods of time than the other samples listed; however, relatively high grafting yields were still obtained. This demonstrates that the process can be achieved quickly, which will have economic advantages for large-scale industrial application of this invention. It is likely that sufficiently high grafting yields can be obtained in 5 minutes or less under appropriate conditions.

Thus, the present invention teaches and demonstrates the effectiveness of a composition comprised of a substrate, preferably fibrous and water-insoluble, to which are attached by non-hydrolyzing covalent bonds a multitude of polymeric chains bearing quaternary ammonium groups. These chains predominantly contain more than one quaternary ammonium group per chain, and preferably have varying lengths and extend varying distances (measured at the molecular level) from the substrate. The present invention also teaches the manufacture of such compositions, where the preferred manufacture includes the steps of dewatering and drying the composition so it is available in a dry (not a hydrogel) form that is more capable of taking up wound exudate.

The present data demonstrates the superior effectiveness of compositions of the present invention compared with siloxane-based polymers such as taught by Blank et al. in U.S. Pat. No. 5,045,322. The '322 patent teaches attachment of monomeric siloxane-based quaternary compounds to super absorptive polymers. The siloxane-based compounds are sensitive to hydrolysis, as noted in the parent application. These siloxane compounds are expected to be more easily hydrolyzed than the acrylate polymers used in the present application. Furthermore, other polymers used in the present invention (such as those based on DADMAC or trialkyl(p-vinylbenzyl) ammonium chloride) are substantially more stable to hydrolysis than the bonds taught in the '322 patent.

In the case of siloxane-based antimicrobial agents, the chemical bonds which are susceptible towards hydrolysis are part of the backbone structure of the polymer. Hydrolysis of even a single siloxane linkage can result in the cleavage of several quaternary units (although the siloxane polymers in such systems are generally only a few units in length). In contrast, in the case of grafted acrylate polymers of the present invention, the grafted chains may be hundreds of units long. The ester linkages which attach the quaternary groups to the polymer backbone are inherently more stable than the linkages in the bulky siloxane quaternary units. Even so, it is possible that the acrylates can be hydrolyzed under extreme conditions. However, since the hydrolyzable group of the acrylate is not in the main chain of the polymer, this will not result in chain cleavage, so the loss under such unlikely, extreme conditions would be limited to a single quaternary unit per hydrolysis event.

Further, the antimicrobial effectiveness of a bulky molecule like the TMS siloxane used by Blank et al. is reduced somewhat by its steric hindrance. Since it can and does fold on itself, the number of such molecules that can be bonded to a given surface is limited as compared to smaller molecules. Further, the fact that the nitrogen atom can be blocked by other atoms in the molecule limits its positive charge density as well. The consequence of this is that the antimicrobial is less effective than one that can be attached to the same surface in greater numbers or density per unit area. Since the net positive charge on the nitrogen atom is related to the effectiveness of the antimicrobial, one that has more exposed positive atoms would theoretically be more effective. This can be shown by comparing the effectiveness of the Blank et al. compounds to any other quaternary compounds that have less steric hindrance. This is demonstrated in the results above, in Tables 2-5. Another consequence is that in the presence of proteinaceous matter such as blood, urine, and tissue cells, the '322 compounds can be blocked more easily than quaternary polymers having a greater concentration of unhindered net positive charges. (See the parent application, PCT/US99/29091, and Table 5.)

A further shortcoming of the siloxane quaternary material disclosed according to Blank et al. is that it only provides a monolayer coverage of the surface. That is, the siloxane backbone molecules are not long-chain polymers. It is well known that siloxane chains more than a few units in length are particularly susceptible to hydrolysis, particularly those with bulky substituents such as the TMS monomer utilized in the '322 patent. This hydrolysis results in chain cleavage and loss of soluble antimicrobial. Such reactions occur as a result of cyclization or “back-biting” reactions (see: J. Semlyen, “Cyclic Polymers” Chapter 3, Elsevier, New York, 1986). By contrast, the surface according to the present invention is covered with polymeric chains composed of non-hydrolyzable carbon-carbon bonds, to which are bonded quaternary materials. Polymeric antimicrobials used according to the present invention are more effective than the monomeric antimicrobials described by Blank et al. (see Chen, Z. C., et al., “Quaternary Ammonium Functionalized Poly(propylene imine) Dendrimers as Effective Antimicrobials: Structure-Activity Studies”, Biomacromolecules 1, p473-480 (2000); Ikeda, T., “Antibacterial Activity of Polycationic Biocides”, Chapter 42, page 743 in: High Performance Biomaterials, M. Szycher, ed., Technomic, Lancaster Pa., (1991); Donaruma, L. G., et al., “Anionic Polymeric Drugs”, John Wiley & Son, New York, (1978)). Thus, in order to obtain a high antimicrobial activity, a high surface area base material must be used with the siloxane quaternary materials. The Blank et al. patent describes placing this monolayer antimicrobial treatment onto powders, which are then used to make superabsorbent polymer gels. The powder has a very high surface area, and hence the gels contain a lot of antimicrobial. However, the Blank et al. gels have almost zero mechanical strength, (and must be contained inside some type of matrix in order to form a useable device). In contrast, the modified cellulose fibers of the present invention have inherent mechanical properties which allow them to be directly used as structural devices such as bandages.

A common understanding in the art is that an “enhanced surface area” would not apply to monolayer treatments such as the siloxane system described by Blank et al. That is, an enhanced surface area substrate is needed to achieve high quaternary content. According to the present invention, however, a high quaternary content may be achieved even on low surface area fibers such as cotton because the quaternary materials of the present invention are polymeric. An analogy may be made to the “fuzzy” structure of a pipe-cleaner to describe a single substrate fiber modified by the currently-described method—that is, each “hair” of the pipe cleaner represents a polymer chain which has an antimicrobial group on substantially each monomer that makes up the polymer. The present applicants have actually attempted use of a Dow Corning product (TMS—the same compound described by Blank et al.) to treat fabrics, and have found that a significantly lower amount of quaternary antimicrobial groups could be applied. The bactericidal activity of the TMS treated fabrics was several orders of magnitude lower than the fabrics treated with polymeric quaternary materials of this invention. The inventors further found that the TMS-treated samples became water-repellent. This effect was reported by Blank et al. (see U.S. Pat. No. 5,035,892; column 12, line 57). This impairment of absorbency is undesirable in a product intended for use as an absorbent. Furthermore, the siloxane monomer has a higher MW than the monomers of the present invention. As a result, the effective quaternary material content (number of positively-charged sites per gram of material) is further reduced as compared to that of the present invention. Finally, the present application further discloses use of neutral or negatively charged antimicrobial polymers, which is neither disclosed nor suggested according to Blank et al.

It should also be noted that the mechanism of action of quaternary compounds is directed towards the cell membrane of the target organism. This process has been described as a mechanical “stabbing” (on a molecular level) which causes rupture of the cell membrane. Thus, it is not possible for pathogenic organisms to develop resistance as observed for most antibiotics. The following examples demonstrate the use of the various initiators described above for the formation of graft copolymers between cellulose and quatemary-containing vinyl monomers:

### EXAMPLE 3

This example demonstrates the grafting of quaternary ammonium polymers onto cellulose fabric. A solution of 0.4 gram SPS, 65 mL distilled water, and 20 mL of Ageflex FM1Q75MC ([2-(methacroyloxy)ethyl]trimethylammonium chloride, 75 wt % solution in water, Ciba Specialty Chemicals Corporation) was placed into a 250 mL screw-top glass jar, and then sparged with argon gas to remove dissolved oxygen. One sheet of rayon non-woven gauze fabric (Sof-Wick, manufactured by J&J) was dried, weighed (2.00 grams total), and placed into the above solution. The jar was flushed with nitrogen, capped, and placed into a 60° C. oven overnight. The fabric sample was then removed, thoroughly washed with tap water, and then dried. The final weight of the samples was 2.49. This represents a grafting yield of 19.4%. The sample was bright white in color, and showed no degradation or discoloration. Testing with a 1% solution of fluorescein dye, followed by thorough rinsing left a bright orange color which indicates the presence of quaternary ammonium groups grafted to the fabric surface. The sample was aseptically cut into approximately one inch² square sections. Each sample section was placed in a sterile 100×15-mm petri dish and covered. Escherichia coli (ATCC 15597) were grown in twenty five milliliters of tryptic soy broth (TSB) (Difco Laboratory, Detroit, Mich.) for 16 hours at 37° C. Each bacterial culture was then diluted a hundred-fold in Fresh phosphate buffered saline (PBS) containing 10% Fetal Bovine Serum (FBS, Sigma, St. Louis, Mo.) to a final concentration of 7.2×10⁷-cfu/mL coli. One-half milliliter of the bacterial suspension was added to each material section. All samples were inoculated in triplicates. The petri dish containing the inoculated sample was then covered and incubated for 18 hours at 36° C. in 100% humidity. Following incubation, the gauze material was aseptically placed into 50-mL conical centrifuge tubes. Twenty-five milliliters of sterile PBS was then added to each tube. The tubes were gently shaken on a rotary shaker (Red Rotor PR70/75, Hoofer Scientific, CA) for 30 minutes. The eluant of samples were then serially diluted thousand and ten thousand-fold by the addition of 1.0 or 0.1-mL of sample to 100-mL of sterile PBS. 0.1-mL aliquots of the diluted samples were then aseptically spread plated onto Tryptic Soy Agar (TSA) (Difco Laboratory, Detroit, Mich.) plates. Additionally, 0.1-mL and 0.33-mL aliquots of the undiluted PBS samples containing the gauze were also aseptically spread plated onto TSA. The plates were incubated for 18 hours at 37° C. The colonies on the respective plates were counted and concentrations were determined. It was found that a greater than 6-log reduction of bacteria was obtained (versus untreated rayon gauze control).

### EXAMPLE 4

The method of Example #3 was used to prepare quatemary-grafted rayon samples. In this experiment, samples were not heated, but instead left at room temperature (25° C.) for various lengths of time. The following results (% grafting vs. reaction time) were obtained: (2 hours—5.5%; 4 hours—13.4%; 69 hours—17.4%).

### EXAMPLE 5

The method of Example #3 was used to prepared quaternary-grafted rayon samples. In this experiment, samples were heated for shorter lengths of time before being removed from the oven and washed. The following results (% grafting vs. reaction time) were obtained: (30 minutes—9.5%; 60 minutes—14.4%; 4 hours—15.4%).

### EXAMPLE 6

The method of Example #3 was used, except the rayon gauze substrate was replaced with bulk cotton (7.08 grams). The following solution was used: 1.5 grams SPS, 210 mL distilled water, and 45 mL Ageflex FM1Q75MC. The sample was heated at 60° C. overnight. The grafting yield was 4.8%. The sample was tested against E. coli bacteria as described in Example #3. A greater than 6-log reduction of viable bacteria was observed.

### EXAMPLE 7

The method of Example #3 was repeated using a 2 hour reaction time at 60° C. In this experiment the step of sparging with argon gas was omitted. The grafting yield was 10.3%.

### EXAMPLE 8

The method of Example #3 was repeated except that 5.05 grams of woven cotton bedsheet material was used as a substrate (1 gram SPS, 70 mL distilled water, and 30 mL Ageflex FM1Q75MC). The grafting yield was found to be 2.8%. The grafted material was tested against E. coli bacteria as described in Example #3. A greater than 6-log reduction of viable bacteria was observed.

### EXAMPLE 9

The method of Example #3 was repeated except that a solution of 3% aqueous hydrogen peroxide (5 mL) was used in place of SPS. The grafting yield was found to be 15.8%.

### EXAMPLE 10

The method of Example #3 was repeated except that 0.50 gram VA-057 (2,2′-Azobis[N-(2-carboxyethyl)-2-methylpropionamidine]tetrahydrate, available from Wako Specialty Chemicals) was used in place of SPS. A 9.5% grafting yield was obtained.

### EXAMPLE #11

The method of Example #3 was repeated, except that a solution of 3% aqueous hydrogen peroxide (3 mL) was used in addition to SPS. A 24.5% grafting yield was obtained.

### EXAMPLE #12

This example illustrates the preparation of a superabsorbent polymer network (SAP). The method of Example #3 was used, except that 0.5 gram of difunctional acrylate crosslinking agent (SR344, polyethylene glycol diacrylate, Sartomer Chemical Co.) was also used. The sample was heated for 2 hours at 60° C., and a solid gel was formed. Excess gel was scraped away from the substrate which was then washed by soaking it in water for greater than 24 hours. The sample was then dried in air. The resulting white fabric sheet was found to be capable of absorbing 25 times its own weight of water.

### EXAMPLE 13

Two grams of mica particles (<38 μm particle size) were placed into a solution of 0.1 g AMBP, 10 g of 65% DADMAC, and 10 g of water, then sparged with argon gas. The mixture was sealed in a jar under argon atmosphere and heated for 90 minutes at 80° C. The mixture was suspended in water (4 L), allowed to settle for several hours, then resuspended in fresh water. After settling overnight, the mica powder wash washed several times in distilled water (50 mL aliquots), and washed by repeated shaking and centrifugation. The powder was then dried in a vacuum oven. Testing of the treated mica with a 1% solution of bromthymol blue dye produced a dark blue coloration after washing. Untreated mica powder tested in a similar manner showed no dye absorption. The powder was tested for antimicrobial activity against E. coli according to the method in the above example. Antimicrobial activity was high, (six log reduction) and no viable bacteria were observed.

The following examples provide detailed written description and enablement for various aspects of the present invention whereby polyionic substrates are created and charged with ionic biologically or chemically active compounds for adherence and sustained release:

### EXAMPLE 14

NIMBUS-10™, (a nonwoven rayon gauze material graft polymerized with diallyldimethylammonium chloride (DADAMC), and containing approximately 10 weight % poly(DADMAC)), and SofWick, a commercially available rayon gauze material manufactured by Johnson & Johnson were used as substrates. The NIMBUS material was prepared via modification of the SofWick substrate. Each substrate measured approximately 40 square inches. Substrates were dried at 60° C. for 30 minutes and then weighed. Both samples were trimmed to weigh exactly 1.00 grams each. A 0.5 weight % solution of Cefazolin Sodium USP (Geneva Pharmaceuticals) was prepared. Each sample was placed in a 50 mL screw-cap polypropylene centrifuge tube, along with 30 mL of the Cefazolin solution and the tubes were placed on a rotating agitator for 3 hours. The samples were removed from the solutions, then squeezed to remove excess solution, dried at 60° C. for 2 hours, then weighed. The NIMBUS sample weighed 1.05 grams, whereas the SofWick sample weighed 1.00 grams. The gravimetric analysis indicated that substantially more drug was absorbed by the NIMBUS sample, compared to the untreated rayon substrate. The extraction liquid was saved for analysis (Solution E1—NIMBUS; Solution F1—SofWick).

The dried samples were placed in separate 50 mL centrifuge tubes containing 25 mL of distilled water, then placed in the rotating agitator overnight at room temperature. The samples were then removed, squeezed to remove excess solution, dried at 60° C. for 2 hours, then weighed. The NIMBUS sample weighed 1.03 grams, and the SofWick sample weighed 0.98 grams. Gravimetric analysis indicated that only a portion of the bonded drug had been released from the NIMBUS sample. The extraction liquid was saved for analysis (Solution E2—NIMBUS; Solution F2—SofWick). This extraction procedure was repeated four additional times to yield two series of six extraction liquids (NIMBUS: E1-E6; and SofWick F1-F6).

The extract solutions were tested for antimicrobial activity by placing single 20 microliter drops of the solutions at marked locations on an agar culture plate spread with ˜3×10³ CFU (continuous lawn) of E. coli bacteria. Plates were incubated overnight at 37° C., and the diameter of the “zone of inhibition”, or “ZOI” was measured. The size of this zone corresponds to the antibacterial activity of the extract solutions. Results are listed below: Solutions E-1 and F-1 were discarded without analysis, since they were likely to contain non-bonded drug. Solution F-3 showed no inhibition of bacterial growth, indicating that all of the drug had been removed in 3 or less washings, thus further samples in this series were not analyzed.

| Sample: | ZOI diameter (mm) |
|---|---|
| Cefazolin 1% | 40 |
| Cefazolin 0.1% | 18 |
| Cefazolin 0.01% | 0 |
| E-2 (NIMBUS) | 23 |
| E-3 | 22 |
| E-4 | 23 |
| E-5 | 18 |
| E-6 | 16 |
| F-2 (SofWick) | 21 |
| F-3 | 0 |

The superior binding, and controlled-release properties of NIMBUS for the Cefazolin drug versus untreated SofWick are clearly demonstrated.

### EXAMPLE 15

This example is similar to Example 14 (above), except that Penicillin G Potassium (PG) (Squibb-Marsam) was used as the drug, and antibacterial efficacy was tested against Staph. aureus instead of E. coli. Samples (NIMBUS: Sample “G”, and unmodified SofWick: Sample “H”) were soaked in 25 mL of 5% PG solution for 2 hours, then squeezed to remove excess solution, dried and weighed. Samples were then washed with 25 mL of distilled water for one hour, then dried and weighed. These wash solutions (G-1 and H-1) were saved for analysis. The samples were then subjected to two additional washings with 25 mL distilled water, without drying in between washings (G-2, G-3, and H-2, H-3). Samples were then dried and weighed. Extract solutions were tested for antimicrobial activity, and the results are shown below. Extract H-3 was found to have zero antimicrobial activity, and thus further extractions were not performed on sample H. Sample G was then subjected to ten additional extractions with 25 mL of distilled water, and only dried and weighed between extractions G8 and G9, and after G13. All extracts were tested for antimicrobial activity, and results are reported below.

| | Sample H (SofWick) | | Sample G (NIMBUS) |
|---|---|---|---|
| Initial sample weight: | 0.971 | g | 1.053 g |
| After drug loading: | 1.058 | | 1.269 g |
| After washing 1x: | 0.979 | g | 1.177 g |
| After washing 3x: | 0.973 | g | 1.147 g |

| | | |
|---|---|---|
| After washing 8x | n.d. | 1.120 g |
| After washing 13x: | n.d. | 1.090 g |

| Solution | ZOI diameter (mm) |
|---|---|
| Penicillin G (1.0%) | 56 |
| Penicillin G (0.1%) | 44 |
| Penicillin G (0.01%) | 0 |
| G-1 (NIMBUS) | 56 |
| G-2 | 48 |
| G-3 | 46 |
| G-4 | 46 |
| G-5 | 46 |
| G-6 | 46 |
| G-7 | 46 |
| G-8 | 46 |
| G-9 | 46 |
| G-10 | 46 |
| G-11 | 46 |
| G-12 | 46 |
| G-13 | 46 |
| H-1 (untreated SofWick) | 56 |
| H-2 | 48 |
| H-3 | 0 |

The NIMBUS sample clearly absorbs more penicillin than the untreated rayon substrate, and binds and releases aliquots of the drug, even after thirteen extractions with distilled water.

### EXAMPLE 16

A repeat of the method of Example 15 was used, except that the extraction solvent used was phosphate buffered saline (“PBS”, pH=7.4, Fisher Scientific), and samples were not dried and weighed between extractions. Samples of NIMBUS (Sample I, initial weight=1.024 g) and SofWick (Sample J, initial weight=1.011 g) were each soaked in 20 Ml of ˜4% penicillin G solution overnight, and excess solution was removed by squeezing. Samples were then washed with 25 mL distilled water for one hour with agitation, squeezed to remove excess liquid, and then subjected to five sequential extractions using 25 mL of PBS for one hour at room temperature. Samples were squeezed to remove excess solution between extractions. Extracts were tested for antibacterial activity against S. aureus according to the procedure outlined above. The results are summarized below.

| | NIMBUS: ZOI | SofWIck: ZOI |
|---|---|---|
| # of extractions | diameter (mm) | diameter (mm) |
| 1 | 55 | 45 |
| 2 | 50 | 34 |
| 3 | 40 | 11 |
| 4 | 30 | 0 |
| 5 | 10 | 0 |

Again, the results clearly show higher initial drug concentration, and prolonged release from the NIMBUS material. Use of saline as the extractant accelerated release of the drug from the substrate; however, the binding effect is still readily apparent.

### EXAMPLE 17

This example demonstrates the stabilization of pyrithione by a cationic cellulose surface. The method of Example 15 was used, except that sodium pyrithione (“SP”, Acros Chemical) was used instead of penicillin. One gram samples of NIMBUS (sample K), and untreated SofWick (sample L) were each soaked overnight in 25 ml of 0.5% SP solution. Samples were removed and squeezed to remove excess liquid. Samples were then washed in 25 mL of distilled water for one hour with agitation, then squeezed to remove excess liquid. Each sample was subjected to four sequential extractions using 25 mL of distilled water for one hour at room temperature with agitation. Samples were squeezed to remove excess solution between extraction cycles. The extracts were tested for antibacterial activity against S. aureus using the procedure described above. Results are shown below:

| | NIMBUS: ZOI | Sofwick: ZOI |
|---|---|---|
| # of extractions | diameter (mm) | diameter (mm) |
| 1 | 12 | 0 |
| 2 | 11 | 0 |
| 3 | 14 | 0 |
| 4 | 12 | 0 |

SP control solutions exhibited the following ZIO (0.1% SP: 26 mm; 0.01% SP: 12 mm). This example clearly shows the binding and stabilization of SP by the cationic cellulose substrate (NIMBUS).

Having generally described this invention, including the best mode thereof, those skilled in the art will appreciate that the present invention contemplates the embodiments of this invention as defined in the following claims, and equivalents thereof. However, those skilled in the art will appreciate that the scope of this invention should be measured by the claims appended hereto, and not merely by the specific embodiments exemplified herein. Those skilled in the art will also appreciate that more sophisticated technological advances will likely appear subsequent to the filing of this document with the Patent Office. To the extent that these later developed improvements embody the operative principles at the heart of the present disclosure, those improvements are likewise considered to come within the ambit of the following claims.

1. A material comprising a substrate and an enhanced surface area, said enhanced surface area comprising a multitude of non-hydrolyzable, non-leachable polymer chains covalently bonded by non-siloxane bonds to said substrate; wherein said non-hydrolyzable, non-leachable polymer chains comprise a multitude of antimicrobial groups attached to said non-hydrolyzable, non-leachable polymer chains by covalent bonds; and wherein a sufficient number of said non-hydrolyzable, non-leachable polymer chains are covalently bonded to sites of said substrate to render the material antimicrobial, or receptive to avid binding of negatively charged dye molecules, when exposed to aqueous fluids, menses, bodily fluids, skin, cosmetic compositions, or wound exudates, wherein said material has associated therewith a plurality of anionically charged biologically or chemically active compounds.

2. The material of claim 1, wherein said antimicrobial groups comprise at least one quaternary ammonium structure.

3. The material of claim 1, wherein said antimicrobial groups comprise at least one non-ionic structure.

4. The material of claim 3, wherein said at least one non-ionic structure comprises a biguanide.

5. The material of claim 1, wherein said non-hydrolyzable, non-leaching polymer chains have an average degree of polymerization selected from about 5 to 1000, 10 to 500, and 10 to 100.

6. The material of claim 1, wherein said material comprises all or part of a wound dressing, sanitary pad, a tampon, an intrinsically antimicrobial absorbent dressing, a diaper, toilet paper, a sponge, a sanitary wipe, isolation and surgical gowns, gloves, surgical scrubs, sutures, sterile packaging, floor mats, lamp handle covers, burn dressings, gauze rolls, blood transfer tubing or storage container, mattress cover, bedding, sheet, towel, underwear, socks, cotton swabs, applicators, exam table covers, head covers, cast liners, splint, paddings, lab coats, air filters for autos, planes or HVAC systems, military protective garments, face masks, devices for protection against biohazards and biological warfare agents, lumber, meat or fish packaging material, apparel for food handling, paper currency, powder, and other surfaces required to exhibit a non-leaching antimicrobial property and to release over time portions of said biologically or chemically active compound.

7. The material of claim 1, wherein said substrate is comprised, in whole or in part, of cellulose, or other naturally-derived polymers.

8. The material of claim 1 wherein said substrate is comprised, in whole or in part, of synthetic polymers including, but not limited to: polyethylene, polypropylene, nylon, polyester, polyurethane, or silicone.

9. The material of claim 1, wherein said attachment of said non-hydrolyzable, non-leachable polymer to said substrate is via a carbon-oxygen-carbon bond, also known as an ether linkage, a carbon-carbon bond, and mixtures thereof.

10. The material of claim 9, wherein a cerium-containing catalyst, a peroxide containing catalyst, an Azo catalyst, a redox initiator, a thermolabile or photolabile catalyst catalyzes formation of said ether linkage or said carbon-carbon bond.

11. The material of claim 1 wherein said non-hydrolyzable, non-leachable polymer chains are formed by polymerization of allyl- or vinyl-containing monomers.

12. The material of claim 11 wherein said allyl- or vinyl-monomers are selected from the group consisting of: styrene derivatives, allyl amines, and ammonium salts.

13. The material of claim 11 wherein said allyl- or vinyl-monomers are selected from the group consisting of: acrylates, methacrylates, acrylamides, and methacrylamides.

14. The material of claim 13 wherein said allyl- or vinyl-containing monomers are selected from the group consisting of: compounds of the structure CH₂═CR—(C═O)—X—(CH₂)ₙ—N⁺R′R″R′″//Y⁻; wherein, R is hydrogen or methyl, n equals 2 or 3, X is either O, S, or NH, R′, R″, and R′″ are independently selected from the group consisting of H, C1 to C16 alkyl, aryl, arylamine, alkaryl, and aralkyl, and Y− is an acceptable anionic counterion to the positive charge of the quaternary nitrogen; diallyldimethylammonium salts; vinyl pyridine and salts thereof; and vinylbenzyltrimethylammonium salts.

15. The material of claim 14 where said allyl- or vinyl-containing monomers are selected from the group consisting of: dimethylaminoethyl methacrylate:methyl chloride quaternary; and dimethylaminoethyl methacrylate:benzyl chloride quaternary.

16. The material of claim 6 wherein said powder is mica.

17. A superabsorbent material for absorbing biological fluids, comprising a substrate and an enhanced surface area, said enhanced surface area comprising a multitude of non-hydrolyzable, non-leachable polymer chains covalently bonded by non-siloxane bonds to said substrate; wherein said non-hydrolyzable, non-leachable polymer chains comprise a multitude of antimicrobial groups attached to said non-hydrolyzable, non-leachable polymer chains by covalent bonds; and wherein a sufficient number of said non-hydrolyzable, non-leachable polymer chains are covalently bonded to sites of said flexible substrate to render the material antimicrobial when exposed to aqueous fluids, menses, bodily fluids, or wound exudates; wherein said superabsorbent material is capable of absorbing about 30 or more times its own weight of water or other fluids in a single instance; and wherein said absorbing capacity is the result of branching or crosslinking of said non-hydrolyzable, non-leachable polymer chains, wherein said material has associated therewith a plurality of anionically charged biologically or chemically active compounds.

18. The material of claim 17, wherein said antimicrobial groups comprise at least one quaternary ammonium structure.

19. The material of claim 17, wherein said antimicrobial groups comprise at least one non-ionic structure.

20. The material of claim 19, wherein said at least one non-ionic structure comprises a biguanide.

21. The material of claim 17, wherein said material comprises all or part of a wound dressing, sanitary pad, a tampon, an intrinsically antimicrobial absorbent dressing, a diaper, toilet paper, a sponge, a sanitary wipe, food preparation surfaces, gowns, gloves, surgical scrubs, sutures, needles, sterile packings, floor mats, lamp handle covers, burn dressings, gauze rolls, blood transfer tubing or storage container, mattress cover, bedding, sheet, towel, underwear, socks, cotton swabs, applicators, exam table covers, head covers, cast liners, splint, paddings, lab coats, air filters for autos planes or HVAC systems, military protective garments, face masks, devices for protection against biohazards and biological warfare agents, lumber, meat packaging material, paper currency, powders, and other surfaces required to exhibit a non-leaching antimicrobial or enhanced dye binding properties, and to release over time portions of said biologically or chemically active compound.

22. The material of claim 17, wherein said substrate is comprised, in whole or in part, of cellulose, or other naturally-derived polymers.

23. The material of claim 17 wherein said substrate is comprised, in whole or in part, of synthetic polymers including, but not limited to: polyethylene, polypropylene, nylon, polyester, polyurethane, or silicone.

24. The material of claim 17, wherein said attachment of said non-hydrolyzable, non-leachable polymer to said substrate is via a carbon-oxygen-carbon bond, also known as an ether linkage, a carbon-carbon bond, or mixtures thereof.

25. The material of claim 24, wherein a cerium-containing catalyst, a peroxide containing catalyst, an Azo catalyst, a thermolabile or photolabile catalyst catalyzes formation of said ether linkage or said carbon-carbon linkage, or mixtures thereof.

26. The material of claim 17 wherein said non-hydrolyzable, non-leachable polymer chains are formed by polymerization of allyl- or vinyl-containing monomers.

27. The material of claim 26 wherein said allyl- or vinyl-monomers are selected from the group consisting of: styrene derivatives; and allyl amines or ammonium salts.

28. The material of claim 26 wherein said allyl- or vinyl-monomers are selected from the group consisting of: acrylates, methacrylates, acrylamides, and methacrylamides.

29. The material of claim 28 wherein said allyl- or vinyl-containing monomers are selected from the group consisting of: compounds of the structure CH₂═CR—(C═O)—X—(CH₂)ₙ—N⁺R′R″R′″//Y⁻; wherein, R is hydrogen or methyl, n equals 2 or 3, X is either O, S, or NH, R′, R″, and R′″ are independently selected from the group consisting of H, C1 to C16 alkyl, aryl, arylamine, alkaryl, and aralkyl, and Y− is an acceptable anionic counterion to the positive charge of the quaternary nitrogen; diallyldimethylammonium salts; vinyl pyridine and salts thereof; and vinylbenzyltrimethylammonium salts.

30. The material of claim 29 where said allyl- or vinyl-containing monomers are selected from the group consisting of: dimethylaminoethyl methacrylate:methyl chloride quaternary; and dimethylaminoethyl methacrylate:benzyl chloride quaternary.

31. An inherently antimicrobial composition comprising:
a. a substrate;
b. a coating, layer, or enhanced surface area on said substrate, comprised of a plurality of polymeric molecules of variable lengths bearing antimicrobial groups, wherein said polymeric molecules are covalently, non-leachably bound to said substrate, and wherein said coating, layer, or enhanced surface area exhibits antimicrobial activity due to the presence of said antimicrobial groups; and
c. ionically associated biologically or chemically active compounds which are released from said substrate and coating layer over a period of time.

32. The composition of claim 31, wherein said antimicrobial groups comprise at least one quaternary ammonium structure.

33. The composition of claim 31, wherein said antimicrobial groups comprise at least one non-ionic structure.

34. The composition of claim 33, wherein said at least one non-ionic structure comprises a biguanide.

35. The composition of claim 31, wherein said material comprises all or part of a wound dressing, sanitary pad, a tampon, an intrinsically antimicrobial absorbent dressing, a diaper, toilet paper, a sponge, a sanitary wipe, food preparation surfaces, gowns, gloves, surgical scrubs, sutures, needles, sterile packings, floor mats, lamp handle covers, burn dressings, gauze rolls, blood transfer tubing or storage container, mattress cover, bedding, sheet, towel, underwear, socks, cotton swabs, applicators, exam table coves, head covers, cast liners, splint, paddings, lab coats, air filters for autos, planes or HVAC systems, military protective garments, face masks, devices for protection against biohazards and biological warfare agents, lumber, meat packaging material, paper currency, powders, and other surfaces required to exhibit a non-leaching antimicrobial or enhanced dye binding properties, and to release over time portions of said biologically or chemically active compound.

36. The composition of claim 31, wherein said substrate is comprised, in whole or in part, of cellulose, or other naturally-derived polymers.

37. The composition of claim 31 wherein said substrate is comprised, in whole or in part, of synthetic polymers including, but not limited to: polyethylene, polypropylene, nylon, polyester, polyurethane, or silicone.

38. The composition of claim 31, wherein said attachment of said non-hydrolyzable, non-leachable polymer to said substrate is via a carbon-oxygen-carbon bond, also known as an ether linkage, via a carbon-carbon bond, or mixtures thereof.

39. The composition of claim 38, wherein a cerium-containing catalyst, a peroxide containing catalyst, an Azo catalyst, a thermolabile or photolabile catalyst catalyzes formation of said ether linkage or said carbon-carbon linkage, or mixtures thereof.

40. The composition of claim 31 wherein said non-hydrolyzable, non-leachable polymer chains are formed by polymerization of allyl- or vinyl-containing monomers.

41. The composition of claim 40 wherein said allyl- or vinyl-monomers are selected from a group consisting of: styrene derivatives; allyl amines and ammonium salts.

42. The composition of claim 40 wherein said allyl- or vinyl-monomers are selected from the group consisting of: acrylates, methacrylates, acrylamides, and methacrylamides.

43. The composition of claim 42 wherein said allyl- or vinyl-containing monomers are selected from the group consisting of: compounds of the structure CH₂═CR—(C═O)—X—(CH₂)ₙ—N⁺R′R″R′″//Y⁻; wherein, R is hydrogen or methyl, n equals 2 or 3, X is either O, S, or NH, R′, R″, and R′″ are independently selected from the group consisting of H, C1 to C16 alkyl, aryl, arylamine, alkaryl, and aralkyl, and Y− is an acceptable anionic counterion to the positive charge of the quaternary nitrogen; diallyldimethylammonium salts; vinyl pyridine and salts thereof; and vinylbenzyltrimethylammonium salts.

44. The composition of claim 43 where said allyl- or vinyl-containing monomers are selected from the group consisting of: dimethylaminoethyl methacrylate:methyl chloride quaternary; and dimethylaminoethyl methacrylate:benzyl chloride quaternary.

45. The antimicrobial composition of claim 44, wherein said substrate is selected from the group consisting of: woven or nonwoven flexible matrices, wherein said composition is formed into the shape of a wound dressing and a powder.

46. The antimicrobial composition of claim 44, wherein said coating absorbs aqueous liquids.

47. The antimicrobial composition of claim 44, wherein said substrate is wood, lumber, or an extract or a derivative of wood fiber.

48. A method for the preparation of a non-leaching antimicrobial-coated composition, comprising the steps of:
a. immersing all or a portion of a substrate into a solution comprising a sufficient quantity of monomer bearing at least one antimicrobial group per monomer molecule, and a sufficient quantity of catalyst to sustain polymerization reactions to sufficiently coat said substrate to impart an antimicrobial characteristic;
b. maintaining the contact of said substrate with said solution under acceptable conditions for a sufficient period of time to complete said reaction, wherein said reactions comprise forming polymers of varying lengths, and forming covalent, non-siloxane bonds between the majority of said polymers of varying lengths and binding sites on said substrate;
c. rinsing said substrate sufficiently to remove non-polymerized monomer molecules, non-stabilized polymer molecules, and catalyst;
d. drying said substrate to a desired low moisture content, such that the substrate is not a hydrogel; and
e. contacting the thus prepared substrate with sufficient anionic or cationic biologically or chemically active compound to achieve ionic association between said compound and said substrate.

49. The method of claim 48, additionally comprising the step of maintaining the solution and gases in contact with the solution free of oxygen by sparging with an inert gas.

50. The method of claim 48, wherein said rinsing is with an aqueous solution, and additionally comprising the step of dewatering the substrate after the rinsing step.

51. The method of claim 48 wherein the catalyst is selected from the group consisting of: a cerium salt, a peroxide, a persulfate, an Azo catalyst, and a photolabile or thermolabile catalyst.

52. An antimicrobial-coated composition for destruction of microbes contacting said composition, comprising:
a. a substrate onto which a coating of antimicrobial polymers is bonded;
b. said coating, formed of an effective amount of polymeric molecules having a multiplicity of quaternary ammonium groups, wherein said polymeric molecules are non-leachably and covalently bonded to surface sites of said substrate, wherein said polymers do not form using siloxane bonds, and wherein said coating is absorbent of aqueous liquids, and
c. associated anionic biologically active or chemically active compound;
whereby said multiplicity of quaternary ammonium groups act to destroy microbes coming in contact with said groups as well as to bind and release said anionic biologically active or chemically active compound.

53. A composition comprising mica which has been subjected to coating, grafting, binding or adhesion of a quaternary amine polymer, followed by association of anionic biologically or chemically active compounds with said quaternary amine polymer.

54. The composition according to claim 1 wherein said plurality of anionically charged biologically or chemically active compounds are selected from the group consisting of: antibiotics, analgesics, anti-inflammatories, strong oxidizing agents, matrix metalloproteinase inhibitors, proteins, peptides, fragrances, and antifungals.

55. The composition according to claim 17 wherein said plurality of anionically charged biologically or chemically active compounds are selected from the group consisting of: antibiotics, analgesics, anti-inflammatories, strong oxidizing agents, matrix metalloproteinase inhibitors, proteins, peptides, fragrances, and antifungals.

56. The composition according to claim 31 wherein said ionically associated biologically or chemically active compounds which are released from said substrate and coating layer over a period of time are selected from the group consisting of: antibiotics, analgesics, anti-inflammatories, strong oxidizing agents, matrix metalloproteinase inhibitors, proteins, peptides, fragrances, and antifungals.

57. The method according to claim 48 wherein said sufficient anionic or cationic biologically or chemically active compound to achieve ionic association between said compound and said substrate are selected from the group consisting of: antibiotics, analgesics, anti-inflammatories, strong oxidizing agents, matrix metalloproteinase inhibitors, proteins, peptides, fragrances, and antifungals.

58. The composition according to claim 52 wherein said associated anionic biologically active or chemically active compound is selected from the group consisting of antibiotics, analgesics, anti-inflammatories, strong oxidizing agents, matrix metalloproteinase inhibitors, proteins, peptides, fragrances, and antifungals.

59. The composition according to claim 53 wherein said anionic biologically or chemically active compounds associated with said quaternary amine polymer is selected from the group consisting of antibiotics, analgesics, anti-inflammatories, strong oxidizing agents, matrix metalloproteinase inhibitors, proteins, peptides, fragrances, and antifungals.

60. A method for treating skin ulcers, bed sores, or chronic wounds which comprises contacting said skin ulcers, bed sores, or chronic wounds with a substrate comprising a polyionic polymer bound to said substrate and a sufficient quantity of matrix metalloproteinase inhibitor ionically associated with said polyionic polymer to achieve extended release of said matrix metalloproteinase onto and into said skin ulcer, bed sore or chronic wound to reduce or eliminate endogenous matrix metalloproteinase activity in said skin ulcer, bed sore or chronic wound.

61. A method of treating a wound which comprises contacting said wound with a substrate comprising a polyionic polymer bound to said substrate and a sufficient quantity of antibiotic, analgesic, anti-inflammatory, or combinations thereof, ionically associated with said polyionic polymer to achieve extended release of said antibiotic, analgesic, anti-inflammatory, or combinations thereof onto and into said wound to reduce or eliminate microbial infection, pain, inflammation at said wound site.

62. An article of clothing comprising a bound polyionic polymer charged with oppositely charged ionic fragrances, antibiotics, antifungals, or combinations thereof.
